(19) United States
(12) Patent Application Publication
(10) Pub. No.: US 2024/0245856 A1
(43) Pub. Date: Jul. 25, 2024

(54) Electromechanical Devices for the Burst Release of Indefinitely Stable Dry Powder Drugs

(71) Applicants: Massachusetts Institute of Technology, Cambridge, MA (US); The Children’s Medical Center Corporation, Boston, MA (US)

(72) Inventors: Siddharth Krishnan, Cambridge, MA (US); Daniel Anderson, Framingham, MA (US); Robert S. LANGER, Newton, MA (US); Arnab Rudra, Brookline, MA (US); Nima Khatib, Somerville, MA (US); Laura O'Keeffe, Nanaimo (CA)

(21) Appl. No.: 18/514,373

(22) Filed: Nov. 20, 2023

Related U.S. Application Data
(60) Provisional application No. 63/480,871, filed on Jan. 20, 2023.

(51) Int. Cl. A61M 5/148

(52) U.S. Cl. CPC A61M 5/148; A61B 5/14532

(57) ABSTRACT

Rapidly administered emergency drug therapy represents life-saving treatment for a range of acute conditions including hypoglycemia, anaphylaxis, and cardiac arrest. A miniaturized (e.g., <3 cm³), lightweight (e.g., <2 g), minimally invasive fully wireless, emergency rescue device for the storage and active burst-release of indefinitely stable particulate forms of peptide and hormone drugs into subcutaneous sites for direct reconstitution in interstitial biofluids is disclosed. The device demonstrates a fast (e.g., <5 minutes) therapeutic effect. The device may deliver a drug across fibrotic tissue, which commonly accumulates following in vivo implantation, thereby accelerating systemic delivery. Fully wireless delivery of dry particulate glucagon in vivo is demonstrated, providing emergency hypoglycemic rescue in diabetic mice. Additionally, triggered delivery of epinephrine is demonstrated in vivo. Additionally, disclosed herein is a platform for the long-term in vivo closed loop delivery of emergency rescue drugs.

### CROSS-REFERENCE TO RELATED APPLICATION

This application claims priority to U.S. Application No. 63/480,871, filed Jan. 20, 2023, the entire disclosure of which is incorporated by reference.

### GOVERNMENT SUPPORT

This invention was made with government support under EB032427 and EB031992 awarded by the National Institutes of Health. The government has certain rights in the invention.

### BACKGROUND

Many soluble drugs, ranging from proteins and peptides to antibodies and nucleic acids, suffer from low stability and short half-lives in the presence of water at physiological temperature. These challenges limit long-term storage and require complex solutions such as cold storage or reconstitution prior to delivery. These storage and delivery challenges provide a particular barrier to the development of closed-loop drug delivery systems since these responsive drug delivery devices typically employ liquid formulations.

Current approaches to triggerable drug delivery involve the use of pumps or of polymeric or hydrogel-based materials with release occurring as a result of conformational changes created by exposure to optical, acoustic, or thermal fields. However, these approaches are incompatible with stable, dry-powder forms of drugs and are inhibited by slow, diffusive release kinetics through solid polymer matrices. One advance in this context involves the encapsulation of dry drugs in hermetically sealed silicon wafers with inorganic thin-film capping layers that can be electrochemically or thermally removed for rapid, on-demand release. While promising, this approach can be limited by the formation of a fibrotic capsule around the device, large currents (>1 A) associated with reservoir opening, small dose sizes associated with each reservoir (<50 μg) and a large overall implant size (about 25 cm³) which can reduce patient compliance.

Emergency rescue often involves administering drugs within 20 minutes from the onset of symptoms. However, the formation of dense fibrotic capsules around implanted drug delivery devices presents a significant barrier and reduces drug diffusion. Delays in systemic drug delivery are particularly problematic for the treatment of acute events, such as hypoglycemia or anaphylaxis. Overcoming the fibrotic tissue barrier accordingly represents an unmet need for emergency drug delivery from implanted devices. These challenges are amplified in clinically attractive subcutaneous sites owing to their relatively high rates of fibrotic tissue formation.

Hypoglycemic rescue for patients with Type I Diabetes (T1D) is an example for emergency rescue drug therapy. Prolonged hypoglycemic periods (blood glucose, BG<70 mg dl⁻¹) or short, severely hypoglycemic periods (BG<54 mg dl⁻¹) can lead to complications that include seizures, coma, and death. The timely administration of glucagon, a naturally occurring peptide hormone secreted by pancreatic alpha cells, is the most effective intervention for acute hypoglycemia, resulting in rapid (e.g., <10 minutes) increases in blood glucose concentrations, and preventing the most severe outcomes of prolonged hypoglycemia.

Unfortunately, glucagon suffers from extremely low (e.g., <1 day) stability in solution, necessitating the use of two-part formulations comprising stable lyophilized drug and liquid vehicles, respectively, for reconstitution immediately prior to injection. The resulting complex, multistep process for emergency glucagon administration involves locating and identifying glucagon kits, removing, and handling pre-filled syringes with needles, mixing liquid vehicles with dry powder drugs, and carefully injecting into subcutaneous or intramuscular sites, and can lead to user error. Additionally, these steps cannot be completed independently by the majority of young pediatric patients or patients experiencing acute symptoms of hypoglycemia such as blurred vision, confusion, or loss of consciousness.

Consequently, the potential for acute hypoglycemic events has also led to a secondary set of challenges for patients with T1D and their caregivers, including reduced compliance with insulin dosing regimens and a reduced quality of life owing to a persistent fear of hypoglycemia. The potential for life-threatening emergencies is particularly acute during periods of sleep, and a subset of T1D-related deaths in otherwise healthy patients has been attributed to acute hypoglycemic episodes at night. Many of the stability and delivery challenges outlined above are also relevant to other classes of emergency drugs, such as epinephrine for the treatment of acute cardiac events and anaphylaxis. Addressing these challenges demands a set of technologies for the rapid, automated closed-loop delivery of emergency drugs as potentially life-saving interventions.

### SUMMARY

A miniaturized, wireless device for the storage and closed-loop, triggerable burst release of stable, dry powder drugs can perform emergency rescue across a range of contexts. An inventive device exploits thermal transport through a thin, flexible electronic systems to achieve rapid (e.g., <25 seconds) actuation of a thermally actuated shape memory alloy membrane for opening a sealed reservoir and ejecting a lyophilized form of an emergency drug contained in the sealed reservoir. The overall size (e.g., about 3 cm³) of the device is much smaller than any comparable technology, with a dose (e.g., about 1.2 mg) suitable for emergency rescue in adult humans. The shape memory alloy actuators can open through fully formed fibrotic tissue capsules as an approach to accelerate release.

Systematic benchtop studies reveal that an inventive device can effect rapid burst release in a range of physiologically relevant conditions, compatibility with multiple drugs, and straightforward integration with existing continuous glucose monitor (CGM) systems for fully closed-loop release. In vivo studies of inventive devices in healthy mice with fluorescently labeled insulin and glucagon reveal release, dissolution, and uptake in subcutaneous sites followed by accumulation in the liver, and glucagon release studies in diabetic mice demonstrate rapid (<10 minutes) increases in blood glucose and hypoglycemic rescue. In vivo studies of inventive devices involving lyophilized epinephrine demonstrate increases in heart rate and blood glucose within 5 minutes of release. These results show that inventive minimally invasive devices can store and burst release stable, powder forms of a range of emergency rescue drugs.

In some aspects, the techniques described herein relate to a device including a substrate including a reservoir to hold a substance, a deformable membrane disposed on the substrate and forming a fluid-tight seal over the reservoir, a thermal actuator mechanically and thermally coupled to the deformable membrane, a thermal insulator, thermally coupled to the thermal actuator, to channel heat from the thermal actuator to the deformable membrane and to reduce dissipation of heat from the thermal actuator into tissue surrounding the device, a circuit operably coupled to the thermal actuator, the circuit configured to receive a control signal and, responsive to the control signal, heat the thermal actuator such that the thermal actuator deforms the deformable membrane to open the fluid-tight seal and effect release of the substance from the reservoir, and a power source configured to provide power to the thermal actuator and the circuit.

In some aspects, the techniques described herein relate to a device wherein the device is sized to be injectable or implantable in a human subject.

In some aspects, the techniques described herein relate to a device wherein a first end of the deformable membrane is fixedly attached to the substrate and a second end of the deformable membrane is removably attached to the substrate, such that the deformable membrane deforms by bending such that the second end of the deformable membrane moves away from the substrate.

In some aspects, the techniques described herein relate to a device wherein the substance is substantially in solid form.

In some aspects, the techniques described herein relate to a device wherein the substance is coupled to the deformable membrane, such that deformation of the deformable membrane effects decoupling of the substance from the deformable membrane and subsequent release of the substance from the reservoir.

In some aspects, the techniques described herein relate to a device wherein the substance includes at least 30 μg of an active pharmaceutical ingredient.

In some aspects, the techniques described herein relate to a device wherein the active pharmaceutical ingredient includes at least one of glucagon, insulin, or epinephrine.

In some aspects, the techniques described herein relate to a device wherein circuit includes a flexible printed circuit board (fPCB) fixedly coupled to the thermal actuator, the fPCB having a flexural rigidity selected to withstand a bending strain experienced by the fPCB and/or the thermal actuator during the deformation of the deformable membrane to remain coupled to the thermal actuator.

In some aspects, the techniques described herein relate to a device wherein the substrate is a first substrate and further including a second substrate coupled to the first substrate to form an enclosure containing the thermal actuator, the thermal insulator, the circuit, and the power source.

In some aspects, the techniques described herein relate to a device wherein the thermal insulator includes a silicone foam.

In some aspects, the techniques described herein relate to a device further including a sealant disposed on the deformable membrane to form at least a portion of the fluid-tight seal.

In some aspects, the techniques described herein relate to a device wherein the sealant includes a multi-layer film including a polymer and a hydrophobic wax.

In some aspects, the techniques described herein relate to a device wherein the control signal is provided by a cellular phone or a continuous glucose monitor.

In some aspects, the techniques described herein relate to a device wherein the reservoir is one of a plurality of reservoirs to hold a plurality of substances, the deformable membrane is one of a plurality of deformable membranes, and the thermal actuator is one of a plurality of thermal actuators mechanically coupled to the plurality of deformable membranes.

In some aspects, the techniques described herein relate to a device wherein the plurality of substances includes individual doses of an active pharmaceutical ingredient.

In some aspects, the techniques described herein relate to a method of administering a substance to a human subject with a device including a substrate forming a reservoir holding the substance, a deformable membrane disposed on the substrate and forming a fluid-tight seal over the reservoir, a thermal actuator mechanically and thermally coupled to the deformable membrane, a thermal insulator thermally coupled to the thermal actuator, and a circuit operably coupled to the thermal actuator, the method including disposing the device subcutaneously in the human subject, actuating the circuit to heat the thermal actuator, deforming the deformable membrane with heat from the thermal actuator so as to decouple the substance from the reservoir, and delivering the substance into the human subject via the decoupling of the substance from the deformable membrane.

In some aspects, the techniques described herein relate to a method wherein disposing the device subcutaneously includes injecting the device.

In some aspects, the techniques described herein relate to a method wherein disposing the device subcutaneously includes implanting the device.

In some aspects, the techniques described herein relate to a method wherein the deforming the deformable membrane produces a force sufficient to tear through fibrotic tissue surrounding the device.

In some aspects, the techniques described herein relate to a method wherein the actuating of the circuit is in response to a signal from at least one of a cellular phone or a continuous glucose monitor.

In some aspects, the techniques described herein relate to a method of administering a substance to a human subject, the method including subcutaneously implanting a device containing the substance in the human subject, causing at least a portion of the device to deform with sufficient force to tear through fibrotic tissue surrounding the device, and delivering the substance into the human subject via deformation of the at least a portion of the device.

In some aspects, the techniques described herein relate to a method wherein causing the at least a portion of the device to deform includes heating a deformable membrane of the device above a transition temperature of the deformable membrane.

In some aspects, the techniques described herein relate to a method of manufacturing a device to hold a substance in a body of a human subject, the method including disposing the substance in a reservoir of a substrate of the device and forming a fluid-tight seal over the reservoir, wherein the fluid-tight seal includes a multi-layer film including a polymer and a hydrophobic wax.

In some aspects, the techniques described herein relate to a method wherein the substance is substantially in solid form.

In some aspects, the techniques described herein relate to a method wherein the substance includes at least 30 μg of an active pharmaceutical ingredient.

In some aspects, the techniques described herein relate to a method wherein the device is manufactured to be subcutaneously injected into the human subject.

In some aspects, the techniques described herein relate to a method wherein the device further includes a deformable membrane disposed on the substrate over the reservoir with the fluid-tight seal a thermal actuator mechanically and thermally coupled to the deformable membrane, a thermal insulator, thermally coupled to the thermal actuator, to channel heat from the thermal actuator to the deformable membrane and to reduce dissipation of heat from the thermal actuator into tissue surrounding the device, a circuit operably coupled to the thermal actuator, the circuit configured to receive a control signal and, responsive to the control signal, heat the thermal actuator such that the thermal actuator deforms the deformable membrane to open the fluid-tight seal and effect release of the substance from the reservoir, and a power source configured to provide power to the thermal actuator and the circuit.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A shows an exploded view schematic of a device, highlighting different layers.

FIG. 1B shows optical top-view images of the device with opaque housing, with a U.S quarter dollar coin and a 200 mg ibuprofen pill for scale.

FIG. 1C shows optical bottom-view images of the device in a transparent housing, highlighting the flexible circuit board and battery, with enhanced image of drug-loading reservoir containing a 1 mg glucagon-lactose pill and shape-memory alloy (SMA) membrane.

FIG. 1D shows the drug release mechanism, with the SMA in its closed, sealed state (left) and opened, released state (right), and a close up of the cut out from FIG. 1A.

FIG. 1E shows an outline of emergency rescue chip operation, highlighting mechanisms for device loading, implantation, and triggered drug release.

FIG. 2A shows optical images of the top view of a device with enhanced view (right) demonstrating fully assembled SMA thermal actuator with thermal insulation foam. Scale bar=5 mm (left), 2 mm (right).

FIG. 2B shows optical images (left) and 3D mechanical finite element simulations indicating strain distributions (right) of coupled flexible thermal actuator-SMA system, with 3N of distributed deformation force past the marked hinge point. Scale bar=1 mm.

FIG. 2C shows optical images of section views of the device with thicknesses of device layers for thermal transport from actuator. Scale bar=1.2 mm (left), 0.8 mm (right).

FIG. 2D shows 3D thermal finite element simulations of temperature distributions during operation with 360 mW of thermal actuation power from flexible thermal actuator, with (top) and without (bottom) insulating foam. Temperatures are normalized by the transition temperature of the shape-memory alloy (35° C.).

FIG. 2E shows SMA temperature measured via a mounted negative temperature coefficient sensing element as a function of thermal actuation power, with and without thermal insulating foam when completely submerged in saline at 28° C.

FIG. 2F shows temperature changes from baselines measured on top and bottom external, tissue-contacting surfaces of device during 60 seconds of actuation when submerged in saline solution. Shaded areas represent averages across three measurements.

FIG. 2G shows mechanical actuation force measured as a function of thermal actuation power in dry conditions and submerged conditions with and without foam. Error bars represent standard deviations across three measurements.

FIG. 3A shows a circuit schematic illustrating passive design for one-dose release with wireless triggering via an external primary antenna.

FIG. 3B shows wirelessly harvested voltage (traces) and actuator current (dots) measured before, during (shaded region) and after drug release, with turn-on voltage for MOSFET indicated via dashed line.

FIG. 3C shows circuit schematic illustrating designs with active control via an on-board microcontroller for multi-reservoir multi-dose release, with flow chart indicating algorithm for microcontroller wake up from power-saving deep sleep mode and a table indicating states corresponding to individual reservoir release.

FIG. 3D shows harvested antenna voltage during release from a four-reservoir device via active control design shown in FIG. 3C, with spikes corresponding to release triggering events. The four left arrows indicate active release events, while the rightmost arrow indicates no release due to exhaustion of the reservoirs.

FIG. 3E shows microcontroller current (traces) and thermal actuator current (dots) over a 125-minute experimental period, with microcontroller current during inter-release periods corresponding to deep-sleep modes.

FIG. 3F shows Rhodamine-C concentrations in deionized water solution following incremental release of pills from 4 separate reservoirs via the mechanisms outlined in FIGS. 3C-3E.

FIG. 4A shows time-series fluorescent images of 1 mg Rhodamine-lactose (960 ppm Rhodamine-C by weight) (Rho-Lac) pill directly submerged in 1% agarose tissue mimic.

FIG. 4B shows time series fluorescent images of 1-mg Rho-Lac pill released from emergency rescue chip in 1% agarose hydrogel tissue mimic at time t=0 s. Scale bar=1 mm for both FIG. 4A and FIG. 4B.

FIG. 4C shows bright-field image of release shown in FIG. 4B. captured about 660 s after release.

FIG. 4D shows time-series bright-field images captured before (left two panels) and 4 s after (right two panels) release, demonstrating localized disruption of material during SMA opening process in 1% agarose hydrogel.

FIG. 4E shows distance traveled by fluorescent Rhodamine diffusion front when submerged directly in tissue mimic (lower trace) and when released from emergency rescue chip (upper trace) as a function of time, demonstrating.

FIG. 4F shows normalized concentration profiles as a function of time before and after release (at time=30 minutes) from emergency rescue chip in 4 ml saline solution, of 1 mg Rho-Lac pill, 1 mg Insulin-lactose (4%-96% by weight) (Ins-Lac) pill, and 1 mg glucagon-lactose pill (4%-96% by weight) (GCG-Lac), respectively, with characteristic time constants indicating time to reach 63.7% of final concentration.

FIG. 4G shows circuit schematic for fully closed-loop burst-release with inputs from continuous glucose monitor (CGM).

FIG. 4H shows a closed-loop burst release in glucose-deionized water solution with dilution via infusion of deionized water from syringe pump, triggered by measurements from CGM, with release of 1 mg Rho-Lac pill triggered by 2 successive measurements below 50 mg dl⁻¹.

FIG. 4I shows time series fluorescent images from in vivo imaging systems (IVIS) measured for mice subcutaneously implanted with emergency rescue chips containing 1 mg Insulin-cy7-Lactose (4%-96% by weight) (Ins-Cy7) pills on day 0, with release occurring on day 7.

FIG. 4J shows total photon flux measurements over regions of interest (ROIs) immediately following release demonstrating rapid increase following release. Sample size: n=3 mice, with error bars representing standard error.

FIG. 4K shows IVIS imaging of major organs t=40 minutes after release of 1 mg Ins-Cy7 pills from emergency rescue chips illustrating biodistribution of drug, with total photon flux measurements from each organ indicated below image.

FIG. 5A shows IVIS fluorescence images following release of 1-mg glucagon-lactose (4%-96% by weight) labeled with a fluorescent dye (cy7) (GCG-Cy7) from emergency rescue chip 2 days after subcutaneous implantation.

FIG. 5B shows IVIS fluorescent image of major organs from 2 animals, 40 minutes after GCG-Cy7 release from emergency rescue chip in subcutaneous sites, illustrating biodistribution of drug.

FIG. 5C shows changes in blood glucose concentrations in 60-minutes following wireless 1 mg GCG-Lac and 1 mg lactose release, respectively, from emergency rescue chips implanted in subcutaneous sites in awake STZ-Diabetic BL6 mice, with release following a fasting period (starting blood glucose level 113±1.9 mg/dl across all animals). N=4 for GCG-Lac group and n=3 for Lactose group.

FIG. 5D shows blood glucose concentrations measured periodically during 60-minutes following wireless 1 mg GCG-Lac and 1 mg lactose release, respectively, from emergency rescue chips implanted in subcutaneous sites in awake STZ-Diabetic BL6 mice, with release following a fasting period and the administration of a subcutaneous insulin bolus at t=−90 minutes, demonstrating hypoglycemic rescue. n=3 for each group respectively.

FIG. 5E shows an area under the curve analysis for blood glucose measurements shown in FIG. 5C-5D for a 30-minute period following release.

FIG. 5F shows representative heart rate (HR) measurements on healthy, anesthetized BL6 mice for 30-minute period following epinephrine release (upper trace) and on mice with sham implants (lower trace).

FIG. 5G shows combined HR changes from baseline following epinephrine release (upper trace) and from sham mice (lower trace). n=3 for each group.

FIG. 5H shows blood glucose changes measured in mice shown in FIG. 5G, following epinephrine release (upper trace) and from the sham group (lower trace). For all data shown here, error bars represent ±standard error. Statistical significance between groups is computed via unpaired, 2-tailed t-test. *p<0.05, * *p<0.01.

FIG. 6A shows histological section images of devices embedded in fibrotic tissue following complete opening and rupture (site 1), partial opening (site 2) and undisturbed tissue (site 3). Scale bars=6 mm (combined image), 0.5 mm for enhanced views of sites 1, 2, and 3 following ex vivo opening after a 7-week subcutaneous implantation period in a BL6 mouse.

FIG. 6B shows optical image illustrating complete opening through capsule (site 1 in FIG. 6A). Scale bar=1 mm.

FIG. 6C shows optical and fluorescent images of Rho-Lac pill following 7-week implantation pill revealing stable, intact pill and confined fluorescence signal. Scale bar=1 mm.

FIG. 7A show a schematic illustration of accelerated testing setup involving transparent, optical window in 3D printed housing, 1 mg Rhodamine-Lactose (4%-96% by weight, Rho-Lac) pill, 1% by weight agarose hydrogel and fluorescent/bright field imaging system with SMA and interfacial sealant.

FIG. 7B shows an initial screen involving wax materials water ingress protection.

FIG. 7C shows an accelerated age screen with bright field imaging with beeswax and varying thickness of parylene-C (0.75 μm, 1.5 μm, 3 μm) at 50° C. in agarose testing system.

FIG. 7D shows representative fluorescent images of dry (left) and partially dissolved (right) pill, with the latter owing to encapsulation failure.

FIG. 7E shows normalized fluorescent intensity for three parylene encapsulation thickness. Each data point is the average of 3 samples, with each sample representing the average of 3 regions of interest (ROIs) at fixed points around the reservoir.

FIG. 7F shows the same analysis as FIG. 7E., but with total fluorescent area as a measure of pill dissolution and diffusion. All error bars represent S.E.

FIG. 8A shows schematic illustrations of ultraflexible thermal actuator.

FIG. 8B shows an optical image of assembled thermal actuator with 0201 resistive elements attached via reflow soldering.

FIG. 9A shows an optical image of wired emergency rescue device embedded in 1% agarose tissue mimic hydrogel.

FIG. 9B shows close-up optical images of device when supplied with thermal actuation powers ranging from 1 mW to 360 mW demonstrating complete opening at 360 mW.

FIG. 10A shows infrared thermographs of an emergency rescue device embedded in agarose hydrogel tissue mimic during passive drug storage.

FIG. 10B shows infrared thermographs of an emergency rescue device embedded in agarose hydrogel tissue mimic during opening.

FIG. 11A shows optical images of a range of Lithium-Polymer (LiPo) batteries with across weights, sizes, and overall capacities.

FIG. 11B shows a discharge current as a function of load for different battery sizes.

FIG. 11C shows discharge power as a function of load for different battery sizes.

FIG. 11D shows battery temperature as a function of load for different battery sizes, as measured by thermocouple attached to battery surface.

FIG. 12 shows the open circuit voltage harvested by secondary receiver circuit as a function of distance from two types of primary transmitter, a charging coil (upper trace) and a standard NFC-enabled smartphone (lower trace), with turn on gate voltage for MOSFET that triggers drug release represented via the dashed line.

FIG. 13A shows drawing files for the board outline, top copper layer, bottom copper layer, plated drill holes, and top and bottom coverlay of a flexible printed circuit board suitable for use in an inventive device.

FIG. 13B shows a bill of materials for board.

FIG. 13C shows an optical image of top view of assembled board.

FIG. 13D shows a side view optical image of assembled board with lithium-polymer battery attached to bottom layer.

FIG. 14A shows a CAD model of a top frame, with an aperture for drug release.

FIG. 14B shows a CAD model of a bottom frame, with a reservoir for drug storage and groove for SMA-based sealing.

FIG. 14C shows a combined assembly computer-aided design (CAD) model (left) and optical image (right) of fully assembled device with top and bottom frames mated together.

FIG. 15 shows optical images illustrating assembly process of completed emergency rescue device with transparent frames allowing for visualization of underlying components.

FIG. 16A shows a circuit layout for drug release via flexible thermal actuator-SMA system triggered by wireless operation via BLE-System on chip capable of supplying voltage directly to a MOSFET gate via commands received from a smartphone.

FIG. 16B shows a prototype graphical smartphone application screen for wireless pairing and drug release.

FIG. 16C shows an optical image of prototype reservoir opened via wireless BLE-based triggering.

FIG. 17A shows optical images demonstrating steps for pill formation from lyophilized drugs.

FIG. 17B shows optical images of full (2 mg) pills and half-pills (1 mg) formed for storage in emergency rescue device via steps shown in A., including 4%-96% Rhodamine-Lactose by weight (Rho-Lac), Insulin/cy7-Lactose (Ins-Cy7), 5%-95% by weight Epinephrine-Lactose (Epi-Lac) and 4%-96% by weight glucagon-lactose (GCG-Lac).

FIG. 18 shows a sequence of bright field images during opening of emergency rescue device containing Rhodamine C-Lactose 4%-96% by weight (Rho-Lac) pill in agarose hydrogel tissue mimic, ranging from completely closed (left), open (middle) and partially dissolved (right) 4 minutes after opening.

FIG. 19A shows a side-view fluorescence image of unreleased Rhodamine C-Lactose 4%-96% by weight (Rho-Lac) pill sealed in emergency rescue device embedded in tissue mimic agarose hydrogel.

FIG. 19B shows a fluorescence image of an example device 20 seconds after opening, with the pill ejected via attachment to SMA, demonstrating a change in fluorescence distribution in the tissue mimic agarose hydrogel surrounding the example device.

FIG. 19C shows a bright field optical image of the opened SMA attached to the pill demonstrating active, ejected opening.

FIG. 20 shows a block diagram illustrating control system for fully closed loop integration with continuous glucose monitor.

FIG. 21A shows a schematic of drug dissolution testing.

FIG. 21B shows a standard curve for calibrating glucagon release as a function of absorbance area.

FIG. 21C shows the release ratio, defined as the amount of glucagon dissolved divided by the amount of glucagon in the pill, over time. N=3 and error bars represent S.E.M.

FIG. 22 shows post explantation images following glucagon (left) and lactose (right) release revealing complete opening and drug dissolution.

FIG. 23A shows optical images of freshly reconstituted epinephrine (left) and epinephrine 48-hours after reconstitution (right).

FIG. 23B shows in vivo heart rate measurements following reconstituted epinephrine (48-hours after reconstitution in sterile deionized water) in subcutaneous sites in n=3 BL6 mice.

FIGS. 24A-24C show dose-response epinephrine measurements. Changes in FIG. 24A show heart rate, in FIG. 24B show SpO₂, and in FIG. 24C show pulse-width distension (a measure of blood flow) following release of epinephrine pills at doses of 0.3 mg/kg and 1.5 mg/kg, along with measurements from mice receiving sham implants.

FIG. 25 shows time series optical images demonstrating SMA opening through fully formed fibrotic capsule ex vivo.

FIG. 26A shows an exploded view of a cylindrical form factor for an injectable device.

FIG. 26B shows a sealed, injectable form factor for the injectable device show in FIG. 26A.

FIG. 26C shows a triggerable opening and ejected release of the injectable device shown in FIGS. 26A and 26B.

FIG. 27A shows a wearable device that functions using ejection of a drug into the dermal skin layer.

FIG. 27B shows the wearable device of FIG. 27A after ejection of the drug into the dermal skin layer.

FIG. 27C shows a microneedle array for use with the wearable device of FIGS. 27A and 27B.

FIG. 28A shows an exploded view of a multi-reservoir device.

FIG. 28B shows an assembled view of the device of FIG. 28A.

### DETAILED DESCRIPTION

An approach to addressing challenges associated with drug stability and emergency burst-release delivery from an implanted device is developed herein. An example of direct reconstitution of lyophilized glucagon and epinephrine directly in interstitial biofluid, with rapid therapeutic effect, is disclosed herein. The platform disclosed herein is relatively small compared to other active drug delivery devices and may allow for straightforward outpatient implantation and replacement, including in pediatric patients. The fear of hypoglycemia in pediatric T1D patients represents a barrier to diabetic care that can result in reduced levels of compliance with insulin dosing regimens and a reduced quality of life for patients and their caregivers. Accordingly, the ability to rapidly deliver emergency doses of lifesaving drugs, including during periods of sleep and hypoglycemia unawareness, is expected to have a direct impact on the treatment and care of pediatric T1D patients.

The device allows for the active ejection of rapidly dissolving dry particulates through fully formed fibrotic capsules. The device may be ingestible by or implantable in a human subject (e.g., a patient). The device may allow for the subcutaneous delivery of drugs for emergency rescue applications, potentially overcoming diffusive transport limitations and allowing for timely interventions to address acutely life-threatening conditions. As such, the platform developed herein can be used to address a broad range of unmet needs in emergency drug delivery, including in the treatment of hypoglycemia, anaphylaxis, cardiac arrest, and hypotension. The device may also be integrated with existing digital sensors such as continuous glucose monitors (CGMs) to allow for patient-personalized thresholds for hypoglycemia and other key biomarkers. The triggering mechanism may also be combined with emergent classes of wearable and implantable biosensors capable of tracking a range of biomarkers indicative of acute, life-threatening events including measures of cardiac activity, thermoregulation, and inflammation. The efficiency of the actuation system may be enhanced by reducing power consumption to regimes compatible with fully wireless, battery-free operation (<30 mW) allowing for further miniaturization and including capabilities in transcutaneous drug refilling.

### A Wireless, Burst Release Drug Delivery System:

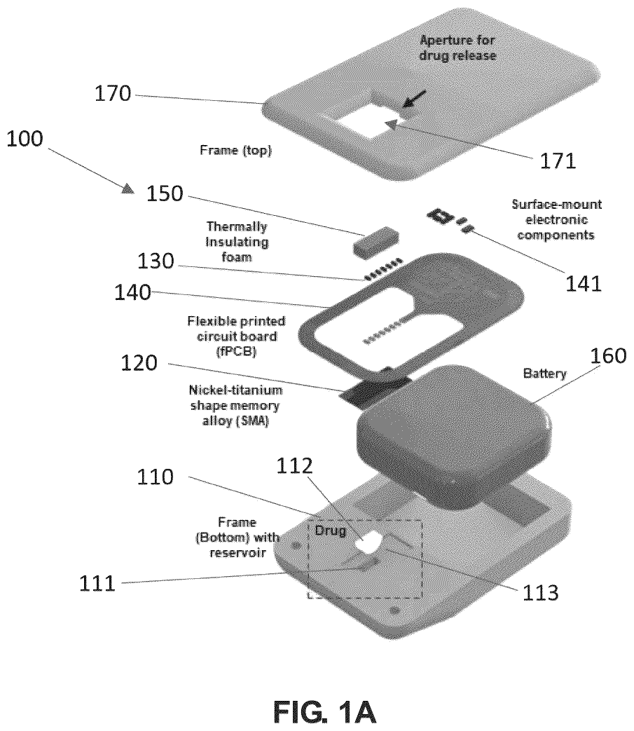

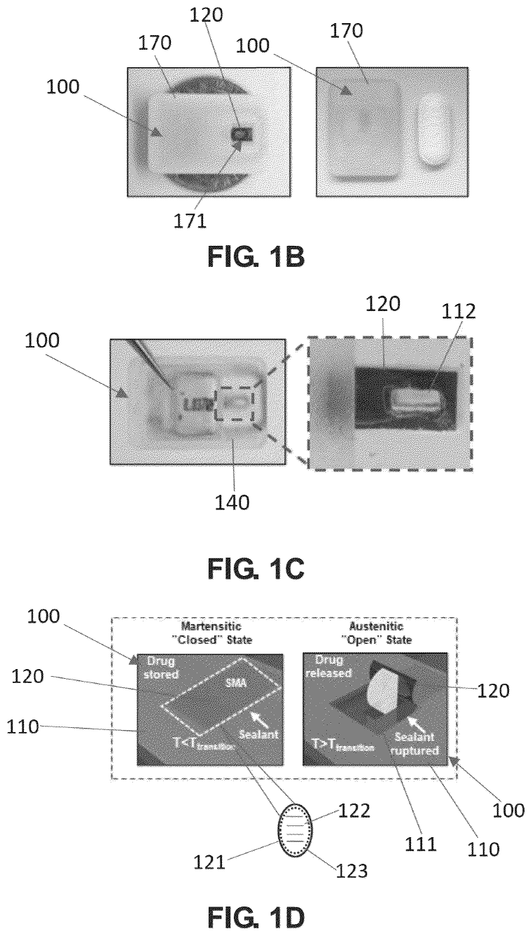

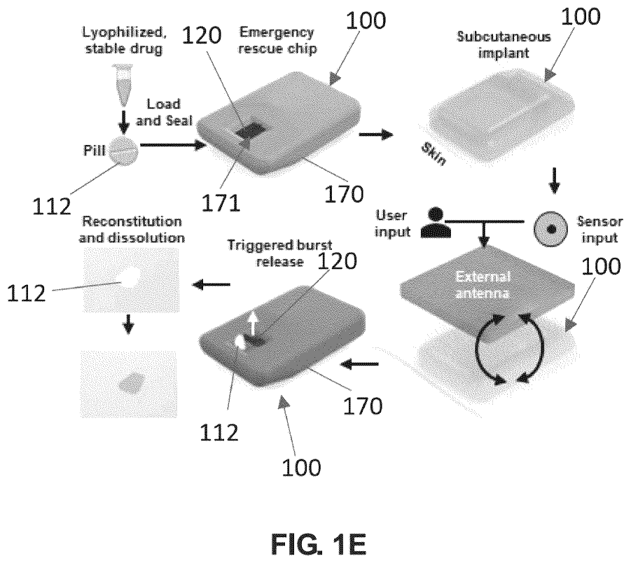

An exploded view schematic of a wirelessly triggered, burst-release drug delivery device 100 is shown in FIG. 1A. Optical images of the device 100 are shown in FIG. 1B, with detailed methods for fabrication and assembly disclosed below. A 3D-printed polymeric frame or substrate 110 includes a slot-shaped reservoir 111 for the storage of a substance (e.g., a lyophilized drug compressed into pills 112), structured inside a recessed feature 113 for sealing as illustrated in FIG. 1C. The substance (e.g., pill 112) may be substantially in a solid form. The pill 112 may be a circular pill, a rectangular pill, and/or any other suitable shape for the device 100.

Sealing of the drug (i.e., substance) occurs via a deformable membrane 120. The deformable membrane may include a flat thin (100 μm) sheet of a nickel-titanium shape memory alloy (SMA). Heating the deformable membrane 120 temperature above its transition temperature (T_{SMA, Transition}) causes a bulk transition from its disordered martensitic phase to a highly ordered, rigid, austenitic phase over short timescales (<10 s). The deformable membrane 120 is programmed into a curved, “open” configuration in its austenitic phase and then cooled to its martensitic phase and deformed into a flat, rolled sheet, which, when combined with an edge-sealant 121, is capable of providing complete sealing in its “closed” state as illustrated in FIG. 1D. The sealant 121 may create a fluid-tight seal over the reservoir 111.

The edge-sealant 121 comprises a multi-layer thin film of a low-permeability polymer 122 (e.g., Parylene-C, Polyisobutylene, Polyimide, Polytetrafluoroethylene, Styrene-Ethylene-Butylene-Styrene thermoplastic elastomer (SEBS), Polystyrene, Polyethylene, and/or another low-permeability polymer)) and a hydrophobic wax 123 (e.g., beeswax, Paraffin, Candelilla), embossed to reduce defect-driven diffusion. The edge-sealant 121 may include up to ten layers of the hydrophobic wax 123, for example, 1, 2, 3, 4, 5, 6, 7, 8, 9, and/or 10 layers of hydrophobic wax 123. Each layer of hydrophobic wax 123 may have a thickness of about 30 μm to about 70 μm, preferably about 50 μm. The hydrophobic wax 123 may be biocompatible, hydrophobic, and embossable or compressible (e.g., able to create defect-free sealing). The hydrophobic wax 123 may also exhibit softening and/or melting at low temperatures to allow for release of the pill 112 upon heating. Preferably the hydrophobic wax 123 is beeswax. The edge-sealant 121 may include up to ten layers of polymer 122, for example, 1, 2, 3, 4, 5, 6, 7, 8, 9, and/or 10 layers of polymer 122. Each layer of polymer 122 may have a thickness of about 500 nm to about 5 μm, preferably about 750 nm. The layer(s) of polymer 122 and hydrophobic wax 123 may be alternated (i.e., one layer of polymer 122 followed by one layer of hydrophobic wax 123) to reduce the potential for defects in multiple layers. The edge-sealant 121 may include up to ten repeating layers wherein one repeating layer includes one layer of polymer 122 followed by one layer of hydrophobic wax 123. Instead of polymer 122, the edge-sealant 121 may include a non-polymer, including but not limited to gold, alumina, hafnia, and/or silicon dioxide. The open configuration is programmed to deform about a single hinge point, using highly localized heating to induce shape change which induces rupturing of the edge-sealant 121, drug ejection, and release.

The device 100 is compatible with various substances including peptides and proteins (e.g., glucagon and insulin), small molecules (e.g., epinephrine) and fluorescent dyes (e.g., Rhodamine-C) and may demonstrate a therapeutic benefit. These classes of drugs are formulated in a similar manner, involving co-lyophilization with an excipient (e.g., Lactose) followed by mechanical pills of based on the desired size and desired dose. The concentration of drug used may range from about 1% to about 100%. The ratio of drug:excipient may range from about 100%:0% to 1%:99%, including all ratios in between. The amount of drug preferably is a therapeutically effective dose of pharmaceutical ingredient (e.g., drug). For example, the amount of glucagon may range from about 40 μg to about 50 mg. A therapeutically effective dose may range from about 30 μg to about 2 mg. Loading into reservoirs, capping with SMA sheets and edge-sealing may allow for the long-term storage of the drug, maintaining its stability in physiological conditions. External wireless triggering, either in closed-loop modes in conjunction with biosensors such as continuous glucose monitors (CGMs) or direct input from users, then triggers particulate drug release directly into subcutaneous sites, followed by rapid reconstitution and dissolution directly in biofluids and uptake into circulation for rapid therapeutic effect. These steps are illustrated in FIG. 1E.

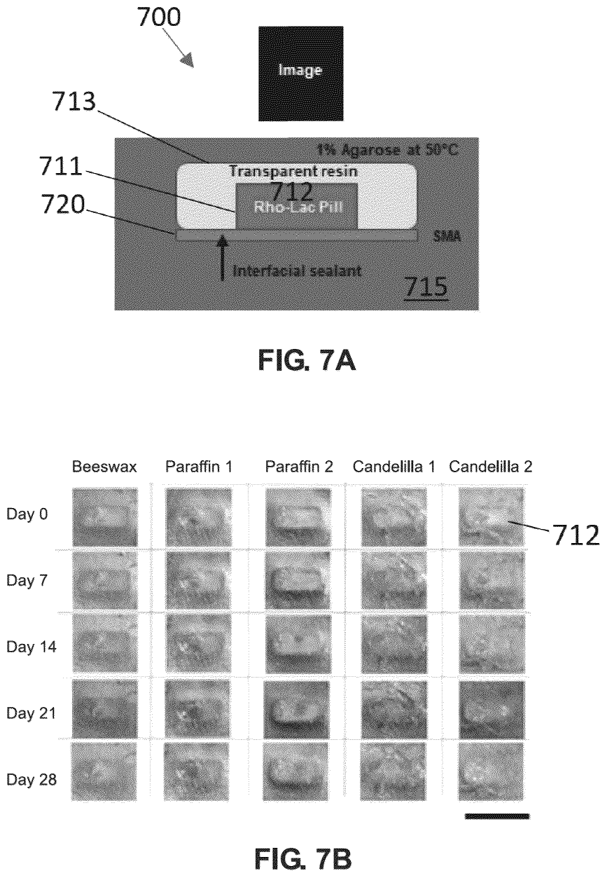

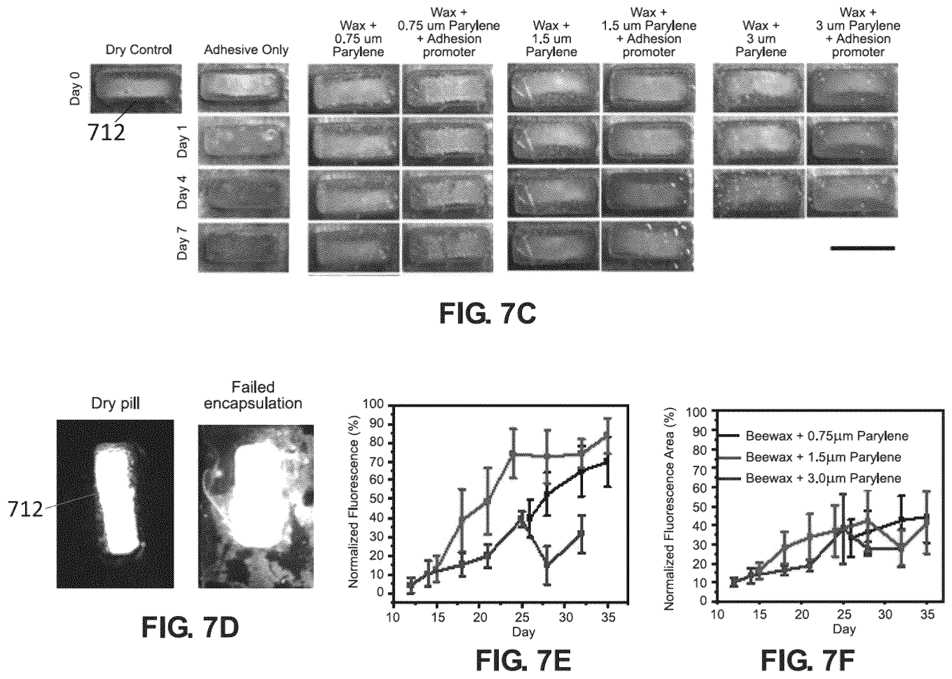

The device encapsulation materials were selected from a multi-material screen based on accelerated rate testing in tissue mimic hydrogel systems at elevated temperatures (FIG. 7A). Parylene-C was chosen for its low water-vapor transmission rate (0.01-0.08 g-mm/m²-day) and a sealing layer of wax 123 may provide hydrophobicity, biocompatibility. A mechanical melting and embossing process may serve to reduce defect-driven diffusion. Accelerated rate testing suggests functional lifetimes of about 120 days for the encapsulation materials disclosed herein. Additionally, a monotonic increase in lifetime may be associated with thicker parylene coating layers (FIGS. 7B-7F), for example, a polymer layer 122 about 3 μm thick. During thermal actuation, a process of wax-softening at elevated temperatures may also serve to promote rapid rupture of the seal and drug release.

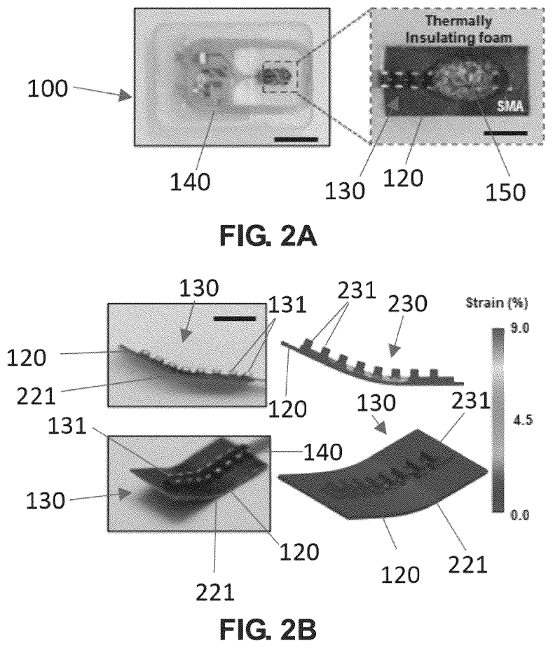

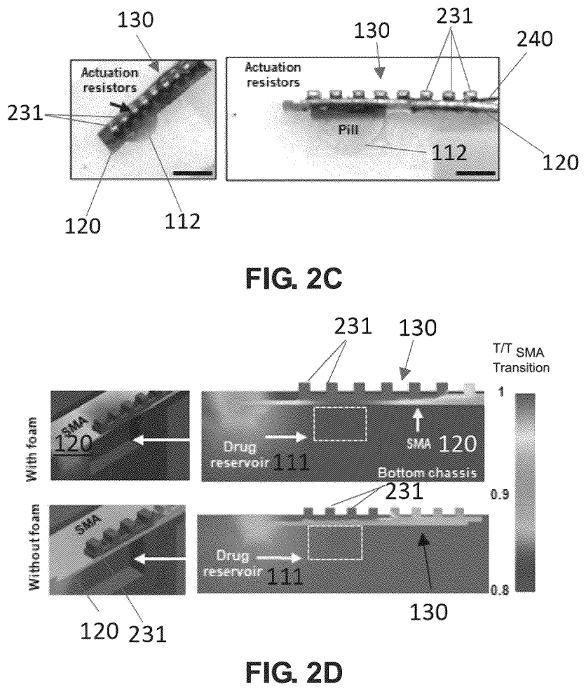

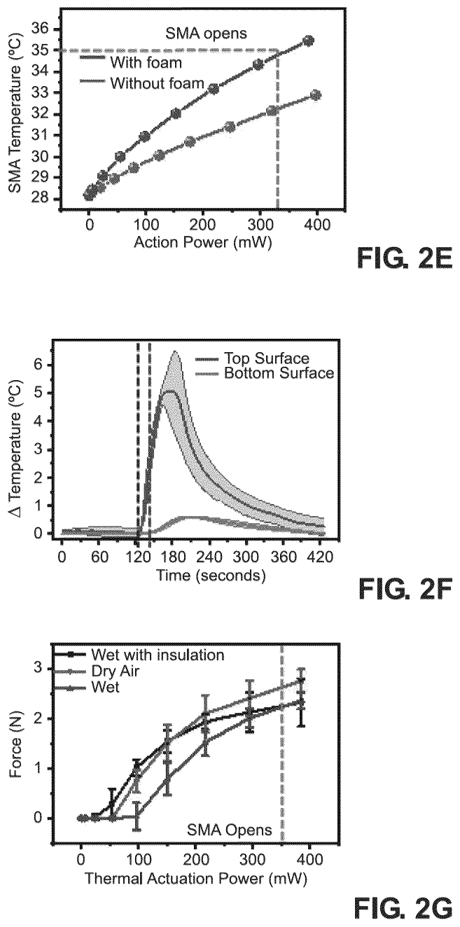

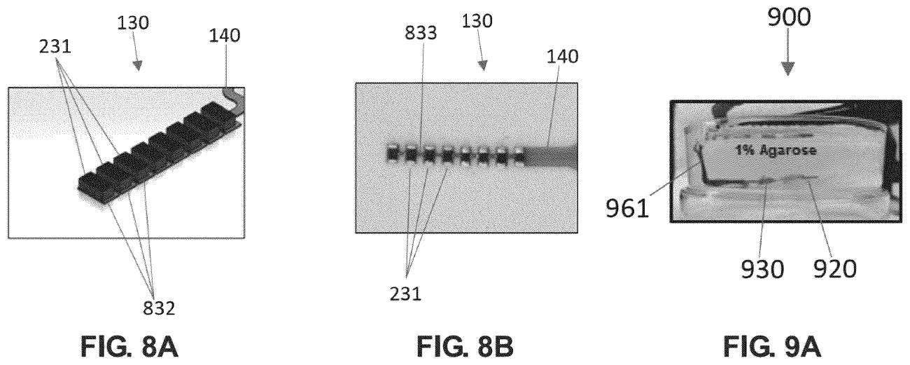

An ultraflexible (bending stiffness about 10⁴ N-m) thermal actuator 130 is disclosed herein and shown in FIGS. 8A-8B. The thermal actuator 130 is thermally insulated with a thermal insulator (e.g., a silicone foam) 150 to induce efficient, rapid shape change in the SMA deformable membrane 120 (FIG. 2A) in wet, physiologically relevant conditions. For the system described herein, T_{SMA, Transition} is chosen to be about 35° C., which corresponds to about 4° C. above measured temperatures at subcutaneous sites (e.g., about 31° C.). The thermal actuator 130 may include an array of linearly arrayed surface-mounted resistive elements 231 electrically connected in series on a circuit 140.

Preferably the thermal insulator (i.e., silicone foam) 150 is applied on top of the resistive elements 231, such that the thermal insulator 150 covers the resistive elements 231 from the top of the device 100 (e.g., from the substrate 170). The thermal insulator 150 may provide insulation for the resistive elements 231 to reduce the amount of heat escaping to the device's surroundings (e.g., tissue), which may lead to inefficient heating of the deformable membrane SMA 120. The thermal insulator 150 may be applied at a thickness of about 500 μm to about 1 mm. Preferably the thermal insulator 150 does not cover the circuit 140 or the deformable membrane 120 to provide greater flexibility to the device 100.

The circuit 140 may include a thin (about 150 μm) flexible printed circuit board (fPCB) (FIG. 1E). The circuit 140 may include one or more surface-mount electronic components 141, including a Bluetooth, Wi-Fi, or cellular antenna and/or a RFID or NFC tag to enable communication with an external device (e.g., a cellular phone or continuous glucose monitor). The Bluetooth, Wi-Fi, or cellular antenna and/or a RFID or NFC tag may also be equipped with two-way communication such that the Bluetooth, Wi-Fi, or cellular antenna and/or an RFID or NFC tag may be used to trigger opening of the device 100 and may also be used to indicate a release event back to an external device (e.g., a cellular phone or continuous glucose monitor). Each individual resistive element may be attached via reflow soldering onto pads 832 that serve as mechanically isolated islands connected via thin bridges 833. Applying a fixed, controlled voltage to the linear, resistive array may result in thermal actuation, with a power P_{Actuator} given by P_{Actuator}=V_{App}²/R_{Array}.

The thin, flexible construction of the linear heating array (e.g., thermal actuator 130) may present two advantages in this context. First, the flexural rigidity of the flexible actuator 130 (about 10⁻⁷ N-m) may be several orders of magnitude lower than that of an equivalent rigid board (about 10⁻² N-m), allowing for coupled deformation of the deformable membrane SMA 120 and the heater (e.g., thermal actuator 130) as a single unit without exceeding yield strains for device materials, as revealed by 3D finite element analysis (FEA) (FIG. 2B). Second, the thermal mass of the underlying circuit (e.g., circuit board) 140 (about 5 mJ cm⁻² K⁻¹) may be significantly lower than that of an equivalent circuit construction from traditional rigid materials (about 100 mJ cm⁻²K⁻¹), allowing for efficient heat transfer to the underlying deformable membrane SMA 120 (FIG. 2C).

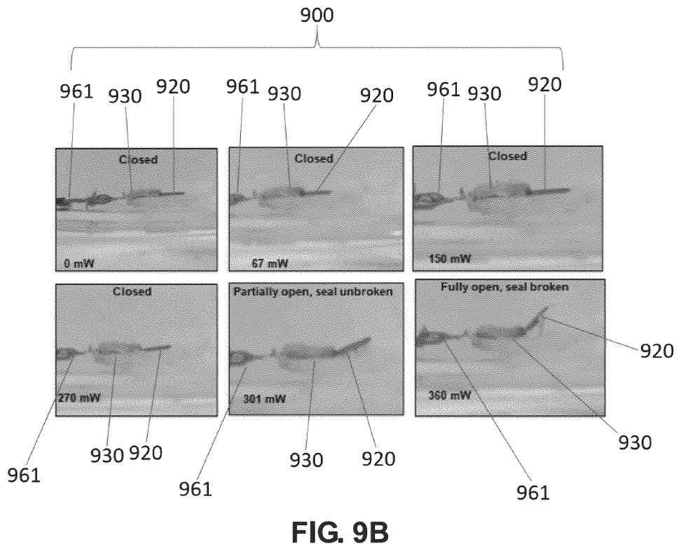

Additional thermal design considerations for operation in wet, physiological conditions reveal the need for thermal insulation. For example, the thermal conductivity of water (k_{water}=0.6 W/m-K) is about 60 times that of dry air (kₐᵢᵣ=0.01 W/m-K). As a result, heat generated in wet environments is dissipated into ambient water, increasing the power demands of the system, as shown in FEA thermal simulations in FIG. 2D. When supplied with less than 350 mW of thermal actuation power (FIGS. 9A-9B), the deformable membrane SMA 120 does not achieve its shape transition temperature, with isotherms largely confined to the superficial circuit (e.g., fPCB) 140 layers. A thermal actuation power of about 350 mW to about 400 mW may be sufficient to enable deformation of the deformable membrane SMA 120 and complete opening of the device 100.

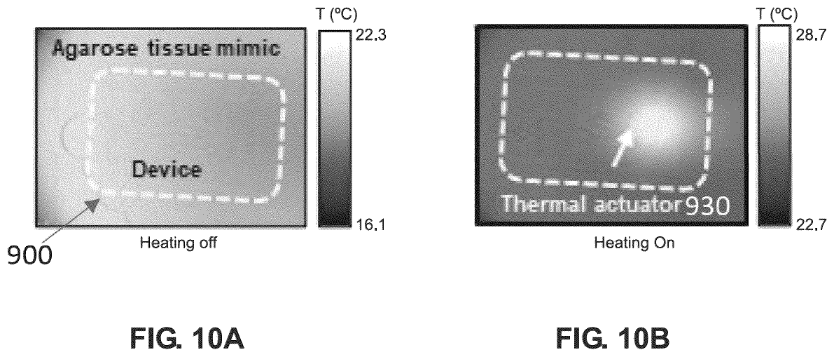

To address this challenge, the linear heating array (e.g., thermal actuator 130) may incorporate a thermal insulator 150 in the form a soft, silicone foam layer with a thermal conductivity (k_{foam} about 0.02 W/m-K) comparable to that of dry air. The addition of a thermal insulator 150 (e.g., a foam) over the resistive elements 231 allows for controlled heating at selected locations to induce shape change, with isotherms that penetrate through the thickness of the deformable membrane SMA 120 (FIG. 2D). The thermal insulator 150 may assist with channeling the transfer of thermal energy into mechanical energy to open the SMA 120 to increase the effectiveness of the device 100 and reducing or preventing dissipation of heat into the surrounding tissue. These results were experimentally validated via temperature measurements directly on the deformable membrane SMA 120 during a power sweep (FIG. 2E). Accordingly, the addition of the thermal insulator 150 may allow for effective interconversion between thermal input power and mechanical work (FIG. 2F), reducing power consumption, reducing battery size, extending battery life, and allowing the deformable membrane SMA 120 to break through both edge-sealant 121 layers and any fibrotic tissue overgrowth. For example, the deformable membrane SMA 120 may be able to tear through fibrotic tissue overgrowth that is about 400 μm thick. The SMA may open in about 25 seconds. The surrounding tissue surfaces in contact with device 100 may reach a temperature about 5 degrees above ambient temperature during the opening of the deformable membrane SMA 120. Additional measurements via calibrated thermistors (FIG. 2G) and infrared imaging (FIGS. 10A-10B) reveal highly confined heating, with tissue-contacting external device surfaces experiencing temperature increases of <5° C. over 60 second thermal actuation periods.

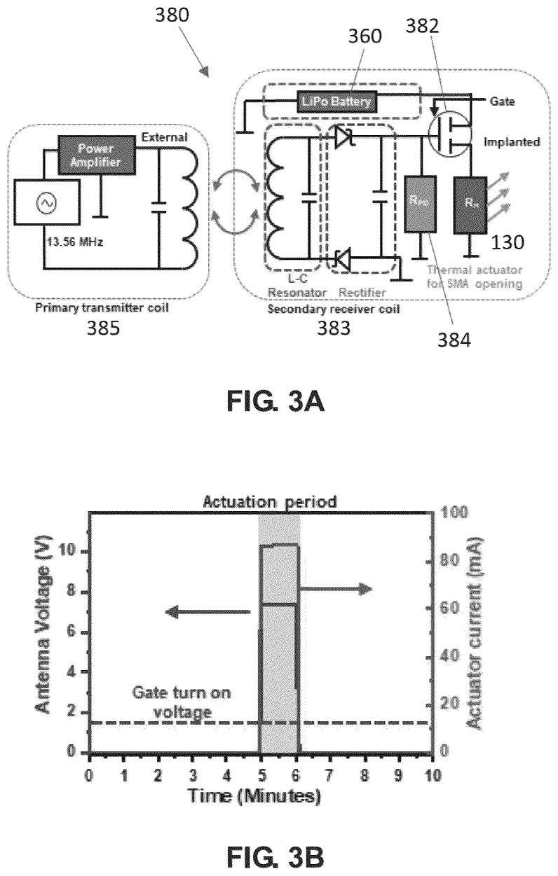

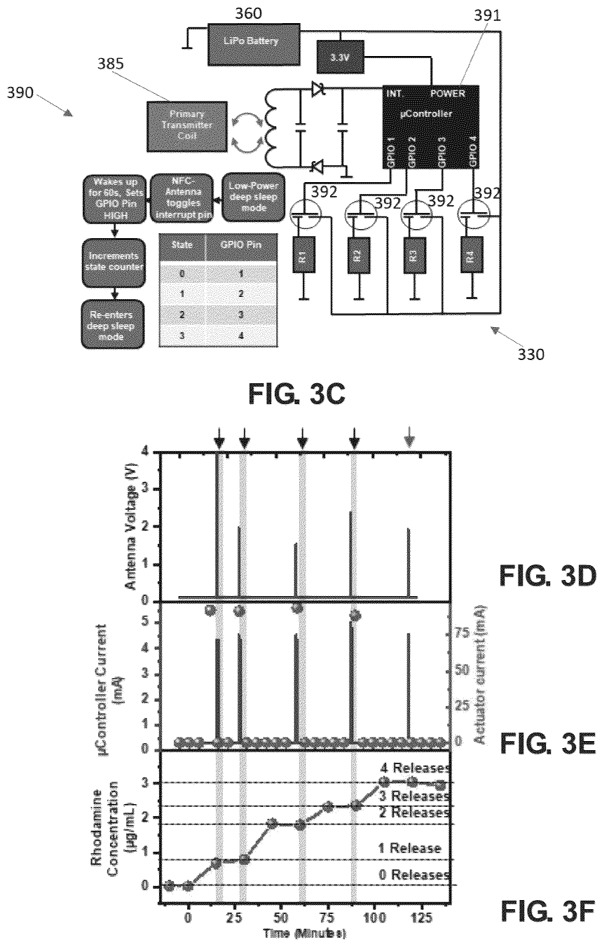

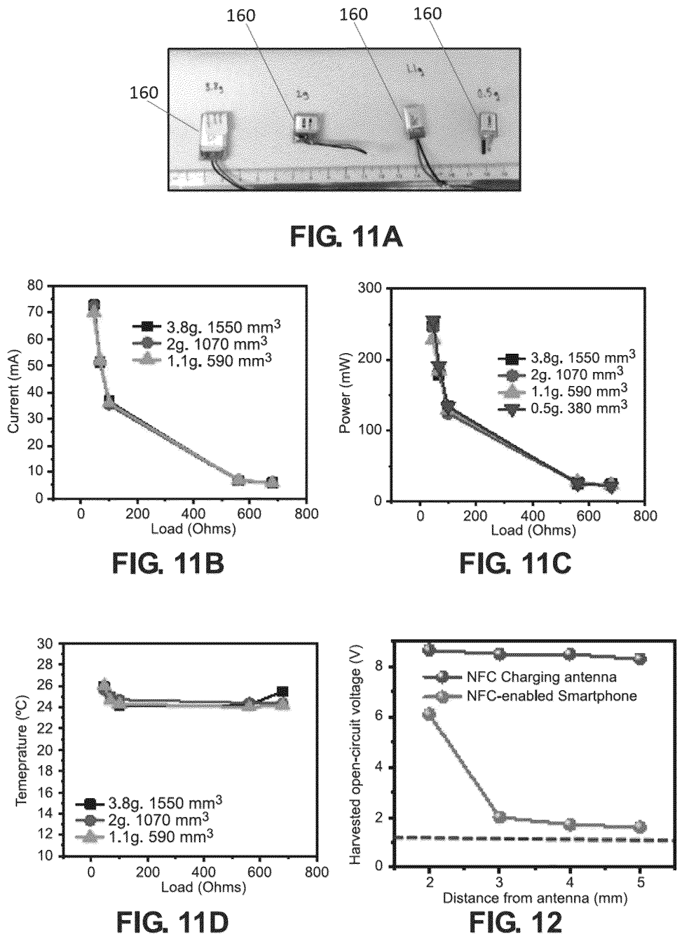

FIG. 3A shows a wireless actuation circuit 380 for a device that stores a single dose and relies entirely on passive components with negligible passive power consumption. The system relies on a small (11 mAh), power source 360 (e.g., a lithium-polymer battery) (nominal voltage 3.8V) for thermal actuation (further power source 360 characterization appears in FIGS. 11A-11D). The power source 360 may range in size. For example, the power source 360 may be about 3.8 g, about 2 g, about 1.1 g, or about 0.5 g. The linear resistive array in thermal actuator 130 comprises four resistors, each 10Ω, wired in series for a total resistance of 40Ω, and a total actuation power of about 360 mW. Alternatively, the device 100 may be battery free instead relying on wireless, battery-free power transfer based on inductive coupling (e.g., using an antenna).

The wireless actuation circuit 380 involves an enhancement-mode metal oxide semiconductor field-effect transistor (MOSFET) 382, with source and drain terminals attached to the resistive heating arrays and batteries, respectively. The MOSFET 382 gate is connected to the voltage output of a resonant inductive coupling circuit designed to harvest power via near-field coupling when in proximity to an externally located alternating current power source operating (FIG. 12). An implanted receiver circuit 383 comprises an L-C circuit, with values chosen to operate at a resonant frequency fᵣₑₛₒₙₐₙₜ of 13.56 MHz given by, fᵣₑₛₒₙₐₙₜ=½π\sqrt{LC} where L and C are the values of the inductor and capacitor, respectively. Operation at 13.56 MHz is chosen for its compatibility with existing NFC communication infrastructure in handheld smartphone and tablet devices, compatibility with both wearable and implantable bioelectronic systems and low specific absorption rates (SAR) in biological tissue. In the default state of the device, a pull-down resistor 384 ensures that the MOSFET gate voltage (V_{Gate}) is 0V, and there is no current to the thermal actuator 130.

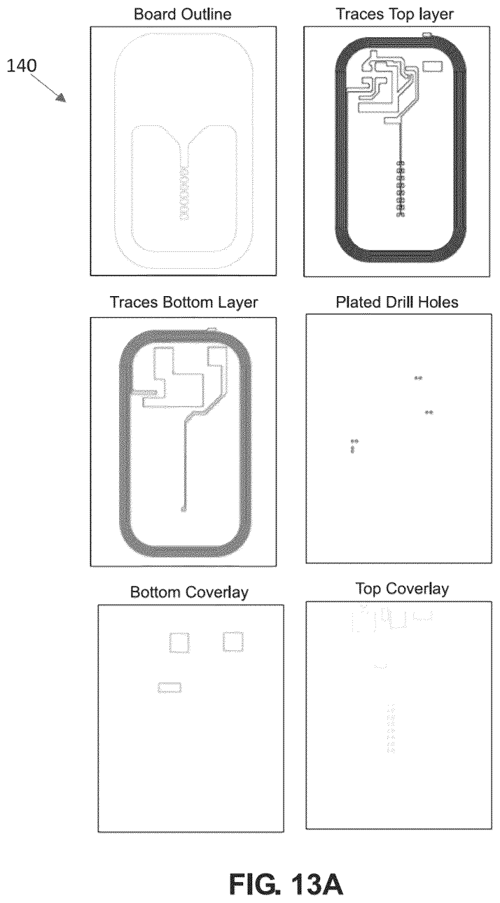

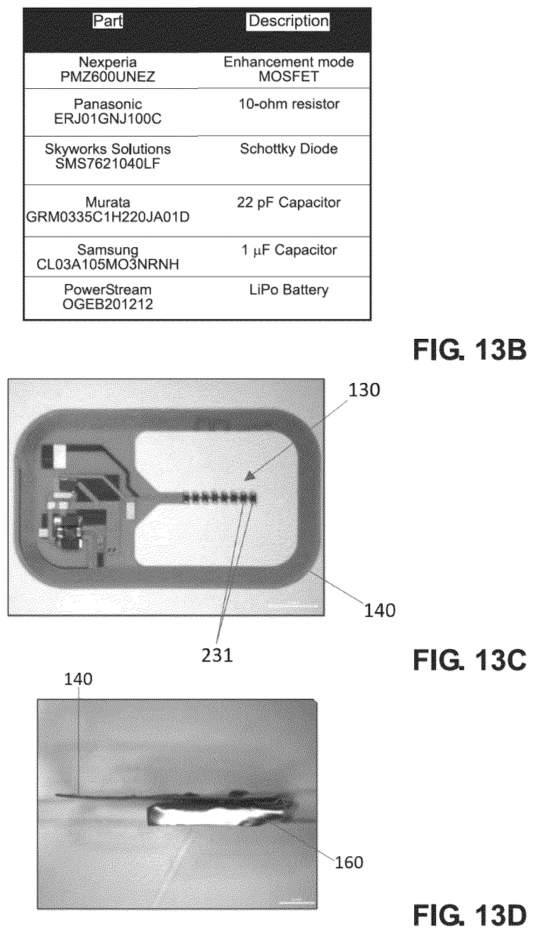

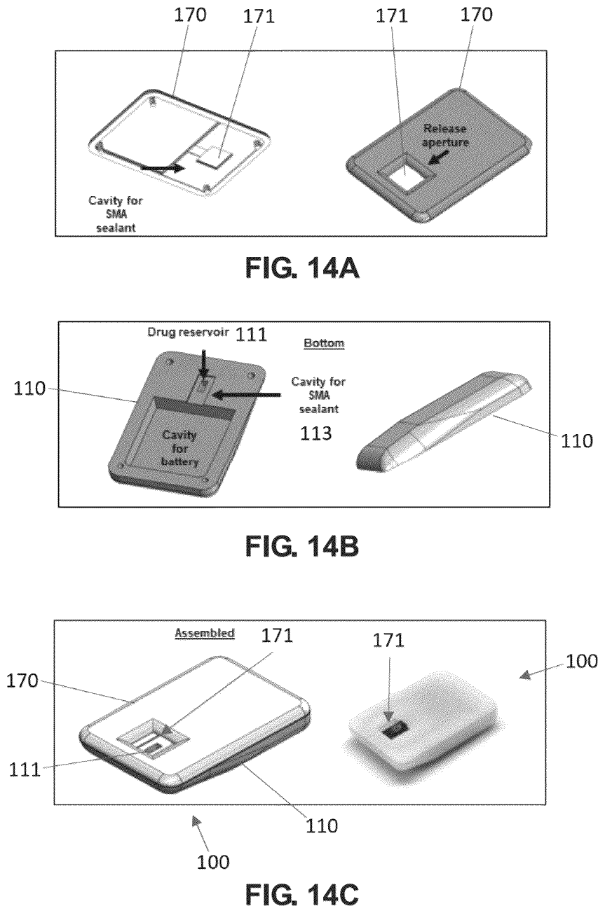

To release the drug, operation of an external primary transmitter coil 385 ensures power transfer to the secondary, implanted receiver coil 383. The received voltage is then rectified and when the resulting DC voltage is supplied to the gate (V_{Gate}>V_{MOSFET Threshold}), current is supplied from the power source 360 to the thermal actuator 130 (FIG. 3B), causing deformable membrane SMA 120 shape change and drug release. This design has two advantages. First, the power consumption during the device's 100 default “off” state is negligible (<1 μA), allowing for extended device lifetimes in vivo. Second, the device 100 can be triggered via direct integration between an external wearable unit and a sensor (e.g., CGM), obviating the need for bulky, implanted devices with on-board storage and control. Circuit schematics, mechanical models and assembly steps for these designs appear in FIGS. 13A-15.

FIG. 3C shows a circuit 390 that expands upon the above concepts to provide options for drug release from multiple, independently controlled reservoirs for the storage of larger doses or combinations of emergency drugs. This circuit 390 includes a lightweight low-power microcontroller 391 (μC) that allows for sequential release. Each reservoir is opened by a MOSFET 392 that controls power to a separate thermal actuator 330, with gate terminals connected to general purpose input/output (GPIO) pins on the μC 391. In this configuration, the μC 391 comprises an internal state variable that is initialized at a value of “0” and is programmed to immediately enter a low-power “deep-sleep” mode that consumes very low (<100 μA) passive current.

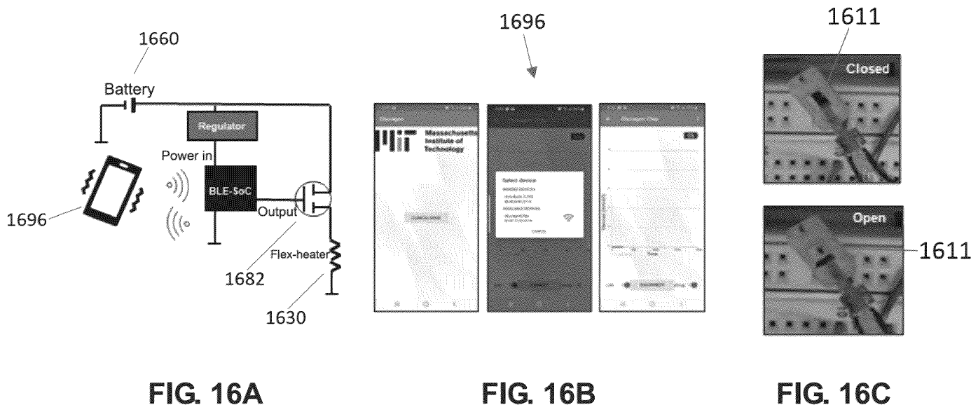

Wake-up events are triggered by voltages supplied directly to interrupt (INT) pins, that trigger subroutines that set a pre-specified GPIO pin from its low to high states for 60 seconds, with the choice of pin determined by the state variable (table in FIG. 3C). The μC 391 then increments the state variable, sets all GPIO pins to their low state and re-enters deep sleep mode. A subsequent wake-up event then repeats the above cycle, but with a new GPIO pin. Each GPIO pin corresponds to a separate reservoir 111, and this approach is broadly generalizable to a large number of reservoirs 111 that is controlled by the number of available GPIO pins on a given μC 391. INT pins are wirelessly triggered by similar resonant inductive coupling approaches. A representative sequence from wireless triggering to multi-reservoir release is shown in FIGS. 3D-3F, for a 4-reservoir device. An expansion of these triggering concepts to wireless technology based on Bluetooth low energy systems on chip (BLE-SoC) is shown in FIGS. 16A-16C.

### In Vitro Release Studies

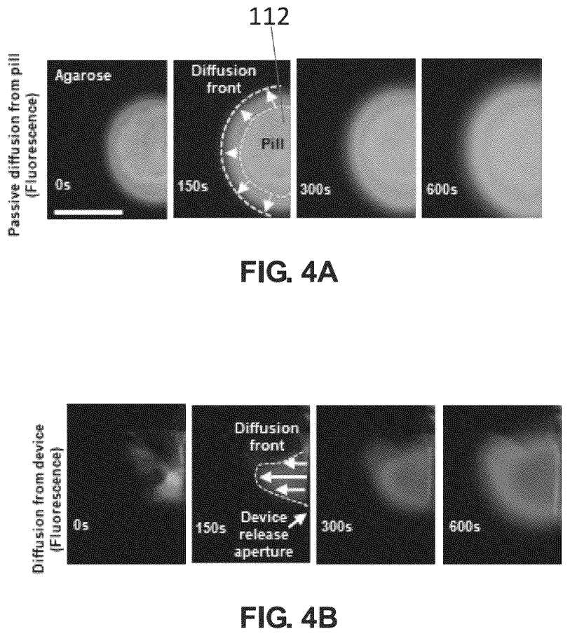

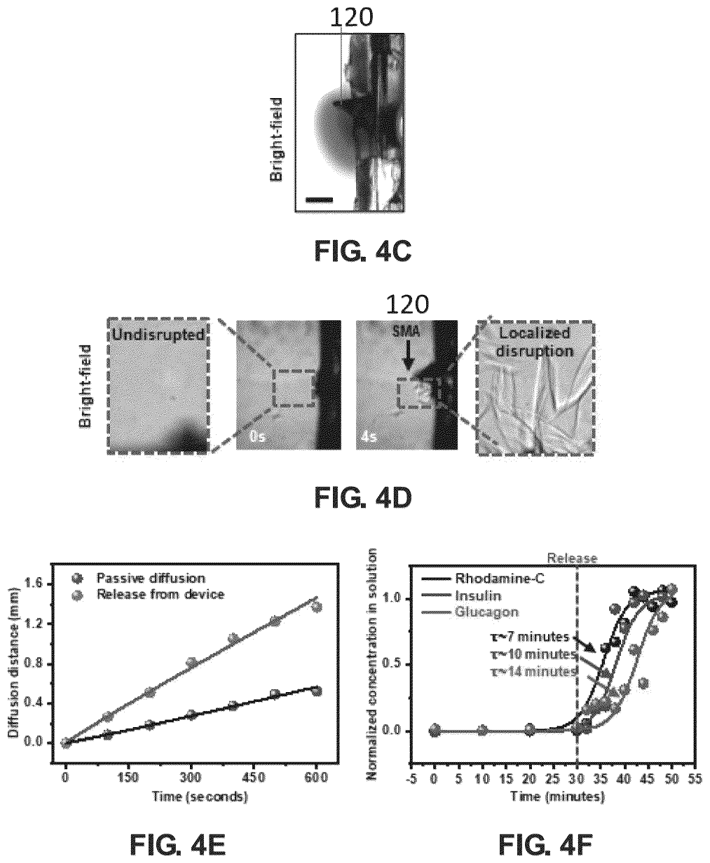

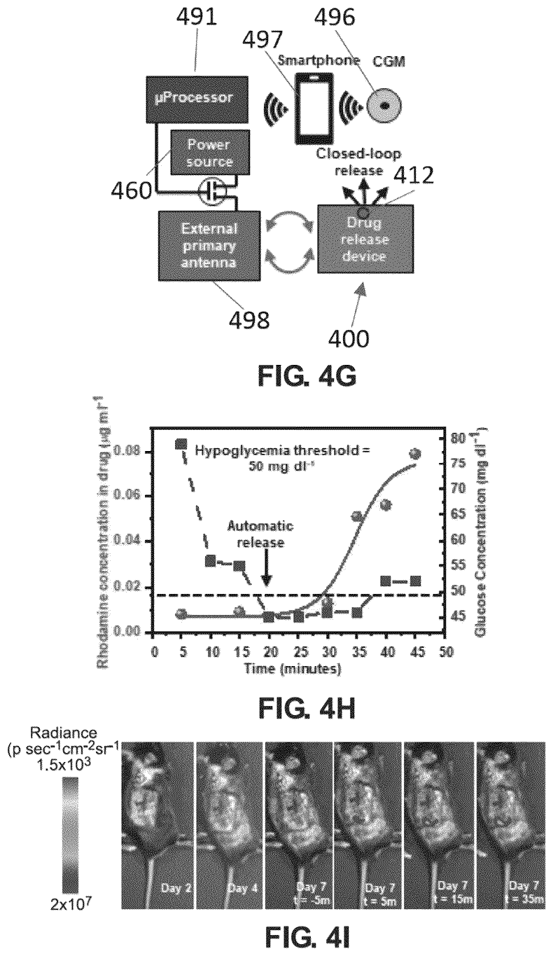

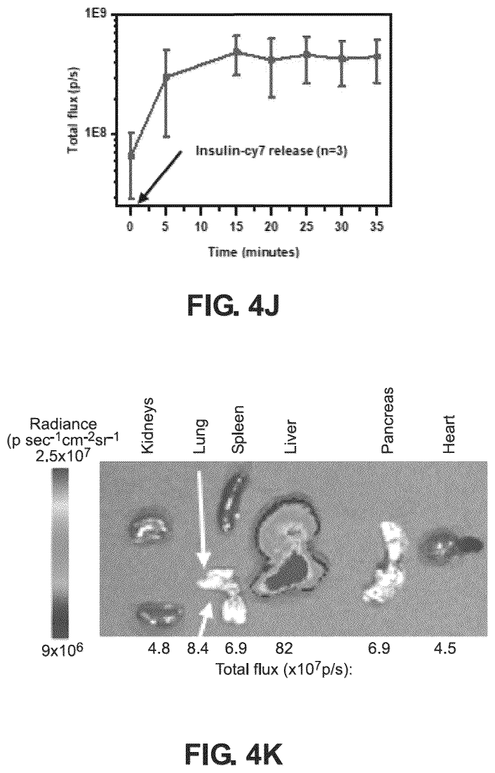

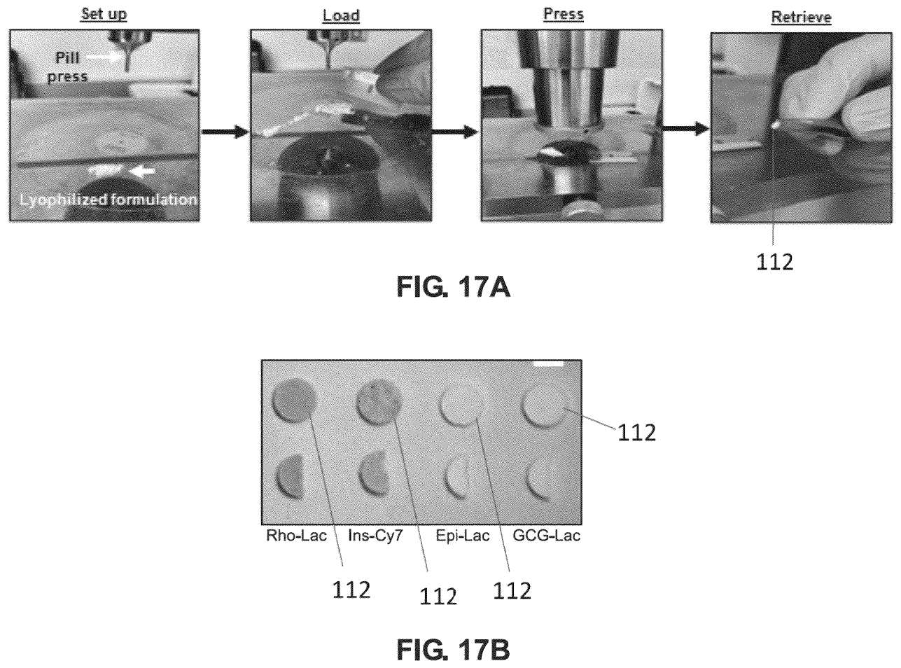

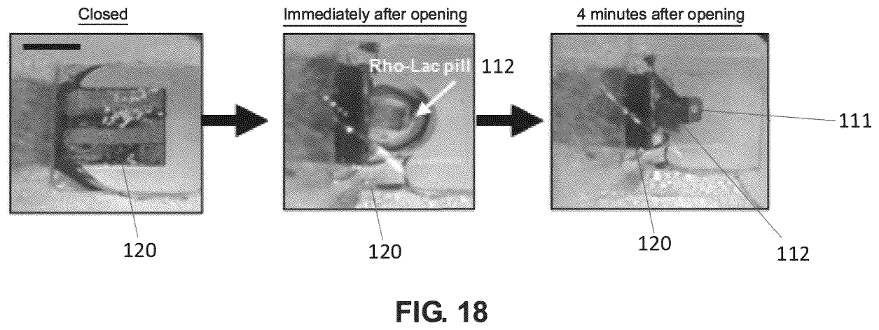

A range of drug formulations and pills (FIGS. 17A-17B) supported systematic in vitro release studies. Experiments involving low-concentration Rhodamine-C in lactose (Rho-Lac, 960 ppm by weight Rhodamine-C) directly embedded in 1% agarose hydrogel materials were used to study passive diffusive drug release, which is shown in FIG. 4A, FIG. 18 in tissue-like materials. The fluorescent diffusion front travels about 400 μm over a 10-minute period comparable to the average diffusion length to a capillary for absorption. In contrast, the diffusion front of fluorescent drugs released from an SMA-actuated device, like the device 100 of FIG. 1A, travels about 1.2 mm over the same time period as illustrated in FIG. 4B, allowing for enhanced uptake and rapid action. Bright-field images captured during the deformable membrane SMA 120 opening period (FIG. 4C) reveal highly localized disruption of the hydrogel tissue mimic, creating channels for enhanced diffusive transport as shown in FIG. 4D.

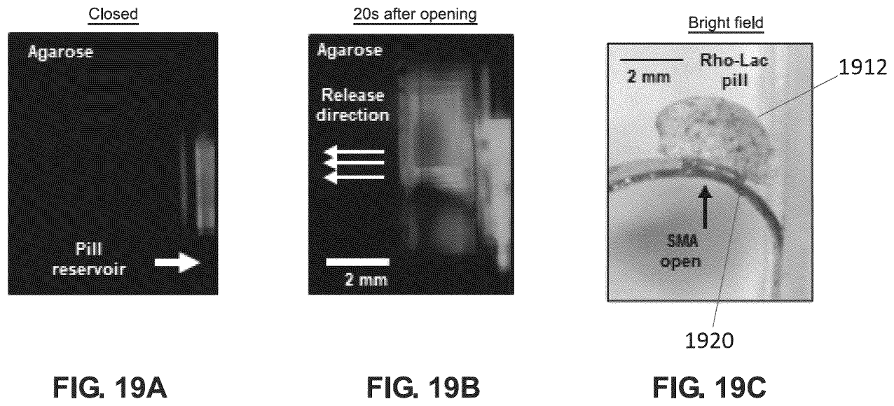

These results suggest a threefold enhancement in diffusive transport from an SMA-actuated delivery system relative to passive diffusion (FIG. 4E). Additionally, these results may suggest mechanistic advantages offered by actuated drug-delivery devices relative to a passive diffusion based systems for emergency rescue applications, where rapid uptake is essential. Further enhancements are possible via entirely ejected release by attaching pill surfaces 1912 directly to SMAs 1920 as shown in FIGS. 19A-19C. A second set of release studies in phosphate buffered saline (PBS) solutions allows for quantitative, time-series concentration measurements based on optical measurements. The release profiles across a range of drugs (e.g., insulin, glucagon, dye), reveal normalized changes in concentrations occurring on timescales of about 7-14 minutes following release (FIG. 4F), illustrating an advantage of these types of devices relative to materials-based approaches that exhibit timescales of several hours.

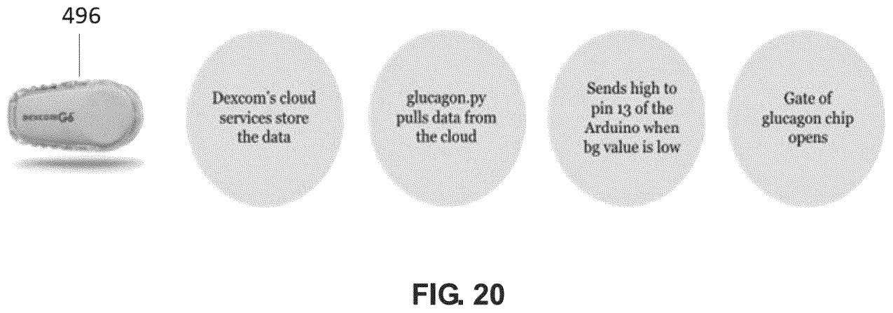

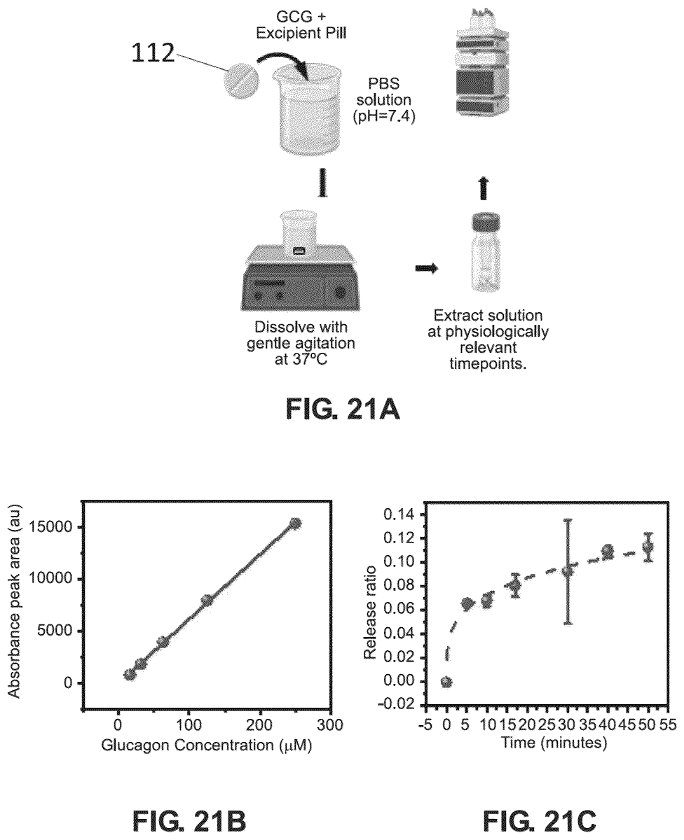

Capabilities in closed-loop burst release enabled by wireless communication between commercially available biosensors and emergency rescue devices represent a feature of this technology. One example is the combination of glucagon release with CGM technology for fully closed-loop glucose-responsive hypoglycemic rescue during periods of sleep or hypoglycemia unawareness and in pediatric patients who are unable to self-administer glucagon. To demonstrate these concepts, a fully integrated approach to receive data from a commercial CGM 496 (e.g., a Dexcom G6) and wirelessly transmit it from a cellular phone 497 to a microprocessor 491 capable of data analysis and processing (FIG. 20) was developed to determine release events based on programmed thresholds for hypoglycemia (FIG. 4G). Benchtop testing in a physiological mimic glucose solution was also performed. Two successive CGM measurements below 50 mg/dl, consistent with thresholds for severe hypoglycemia, trigger microprocessor 491 commands that activate a primary transmitter coil, which includes a power source 460 and results in wireless activation via an external primary antenna 498 of the drug release chip device 400 and burst release of a fluorescently labelled pill 412 (FIG. 4H). Further in vitro release studies based on high-performance liquid chromatography (HPLC) confirm rapid dissolution of clinically relevant dry powder drugs such as Glucagon across a range of loading fractions (FIGS. 21A-21C).

### In Vivo Release Studies

Imaging studies with fluorescently labeled lyophilized insulin-dye conjugates (Ins-Cy7) validate burst release in vivo, over timescales of about 10 minutes. Devices (e.g., device 100) were implanted in subcutaneous sites, with release apertures 171 facing into muscle beds (the complete surgical implantation procedure is disclosed below). IVIS imaging revealed no detectable fluorescence until wireless release 7 days following the implantation procedure, which results in a rapidly expanding fluorescent signal originating from the location of the pill reservoir as shown in FIG. 4I. Fluorescence is detectable within 5 minutes of release, suggesting rapid dissolution and reconstitution in biofluid in subcutaneous sites. Quantitative measurements of regions of interest (ROI) further validate these findings, revealing a roughly tenfold increase in local fluorescence intensity within the first 15 minutes following release (FIG. 4J). IVIS imaging also demonstrates the biodistribution of the subcutaneously released dry-powder insulin, with fluorescence signals confined to the liver (FIG. 4K), consistent with established sites for insulin metabolism.

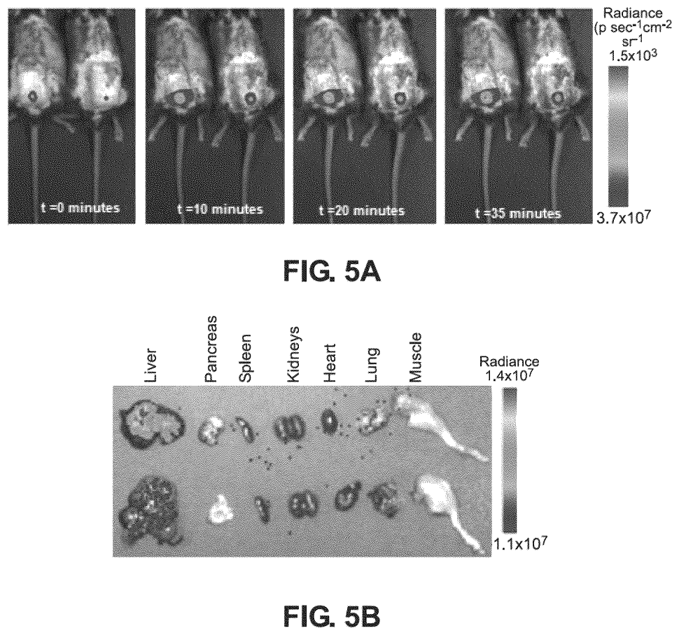

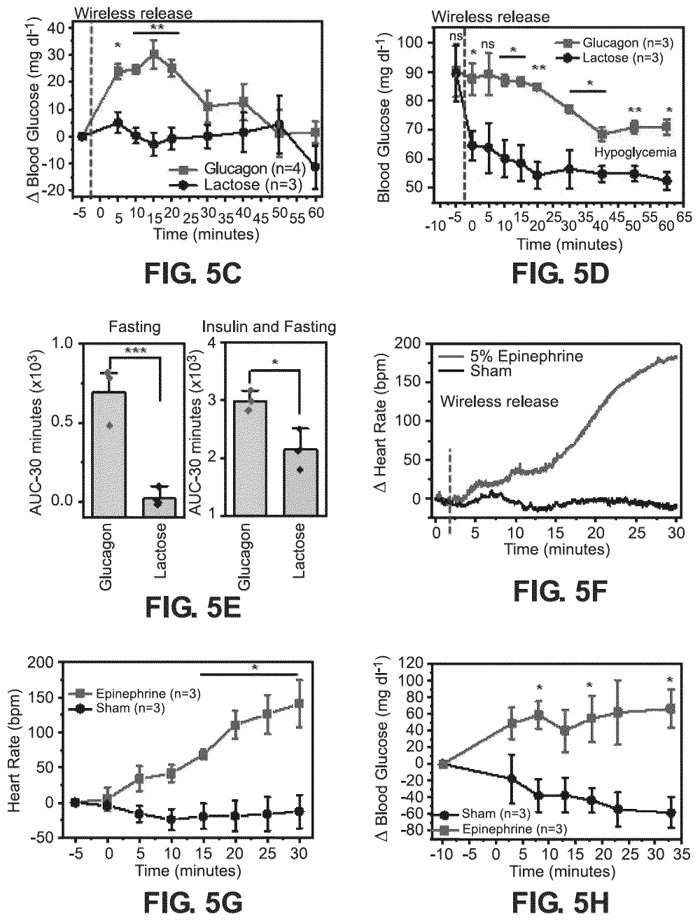

An equivalent set of studies with glucagon conjugated with cy7 (GCG-cy7) reveal similar release profiles as illustrated in FIG. 5A with strong fluorescence visible within the first 20 minutes following release. Biodistribution studies also suggest confinement to liver site (FIG. 5B). Taken together with insulin release studies, these results suggest that dry-powder release from subcutaneous sites may result in rapid dissolution and reconstitution in biofluids, followed by transport to the liver for receptor activation, consistent with trends observed with subcutaneous injections of liquid drug. As shown herein, dry-powder injection directly into subcutaneous sites may function as a mechanism to both maintain long-term drug stability and ensure rapid action following release.

### Emergency Hypoglycemic Rescue in Diabetic Mouse Models:

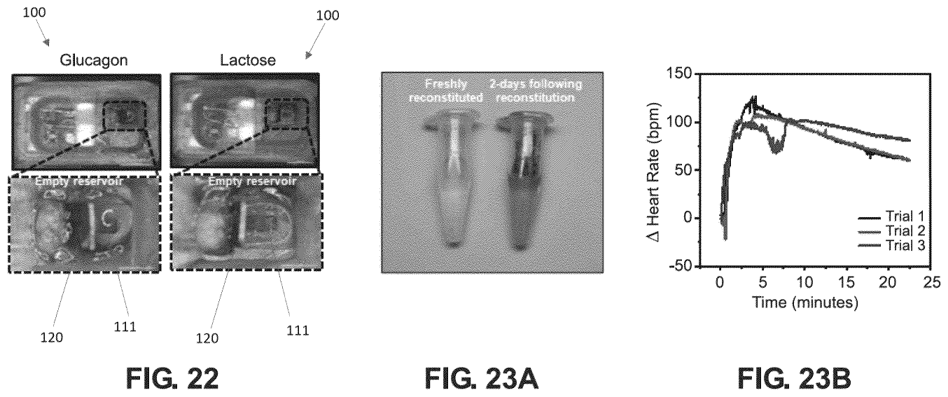

Functional studies involve the release of glucagon-lactose pills (GCG-Lac, 4%-96% by weight, 1 mg total weight) in subcutaneous sites in C57/BL6 mice with chemically induced diabetes via Streptozotocin (STZ) injections. In an initial set of experiments (results shown in FIG. 5C) mice were fasted to induce mild hypoglycemia baseline BG 113±1.9 mg dl⁻¹). Wireless release is triggered at time t=0 minutes by placing the awake, freely moving mice in a modified enclosure capable of providing wireless power at 13.56 MHz. Successive measurements then reveal a rapid rise in BG measurements relative to baselines, with a peak value (30 mg/dl) occurring at tₘₐₓ=15 minutes following release. These BG concentrations are significantly higher than those of a control group loaded with lactose, a non-active drug excipient over a period of 20 minutes following release. Following release, explantation confirmed complete opening and dissolution of the pills (FIG. 22). Notably, strong behavioral differences between the two groups were observed, with the GCG-lactose group exhibiting bright, alert, reactive behavior and the lactose control group exhibiting signs of hypoglycemia such as minimal movement and low reactivity. These results suggest that dry-powder glucagon stored in sealed devices may remain stable in in vivo conditions, followed by rapid release and reconstitution directly in biofluid, as confirmed by both biomarker (BG) measurements and behavioral assessments.

Hypoglycemic rescue is commonly used in cases involving insulin infusions from automated pumps, followed by missed meals and the potential for these incidents represents a barrier to more widespread adoption of automated pump technology. In a second set of studies, designed to capture these conditions, subcutaneous insulin (200 mU) injections were provided to STZ-diabetic mice following a 90-minute fasting period, resulting in a rapid decline in BG concentrations. Wireless GCG-Lac (GCG-Lac, 4%-96% by weight, 1 mg total weight) release from the emergency rescue chip resulted in an average BG concentration of 80±8 mg/dl over the 60 minutes following release, above the threshold for hypoglycemia and significantly elevated relative to BG concentrations measured in animals with lactose controls (average BG: 57±4 mg/dl over 60 minutes following release) (FIG. 5D). Taken together, these results demonstrate hypoglycemia prevention via the subcutaneous release of dry-powder glucagon. While BG measurements provide a point measure of drug delivery efficacy, area under the curve (AUC) analysis over 30-minutes following release provides an integrated measure of BG changes following release. These measurements reveal a significantly elevated AUC in the GCG group relative to lactose controls, in cases involving both fasting and fasting with an insulin bolus, respectively (FIG. 5E).

### Burst Release of Epinephrine for Broad-Spectrum Emergency Rescue Applications:

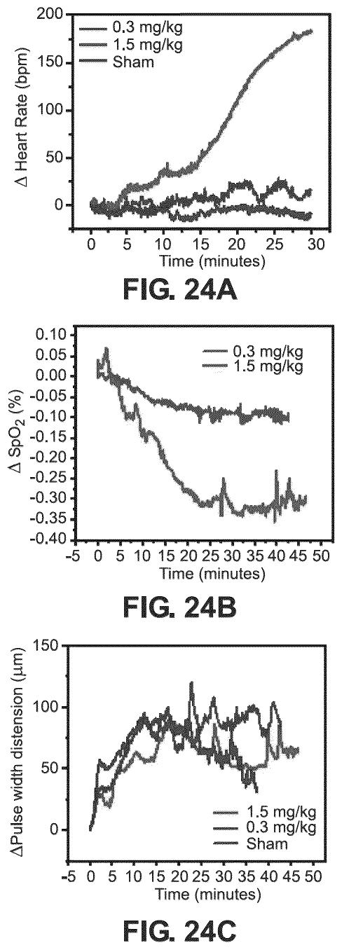

The emergency rescue chip is broadly compatible with lyophilized drug formulations. To test this, studies with epinephrine, a naturally occurring circulating neurotransmitter hormone produced by the adrenal medulla, were performed and used as emergency medication in the treatment of anaphylaxis, cardiac arrest, asthma, and hypotension. Epinephrine release targets adrenergic receptors is accompanied by increases in heart rate (HR). Wireless Epinephrine release (1 mg pill, 5%-95% Epinephrine-Lactose by weight) in subcutaneous sites in anesthetized mice resulted in rapid increase in HR (FIGS. 4F-4G), and BG (FIG. 4H) with increases evident within 10 minutes of release. HR measurements (ΔHR_{Epi}=140±33 bpm) and BG concentrations (ΔBG_{Epi}=66±23 mg/dl) are significantly elevated relative to those of a control with sham devices group (ΔHR_{Sham}=−12±23 bpm, ΔBG_{Sham}=−58±18 mg/dl) 30 minutes after release. Comparisons with subcutaneously injected epinephrine (FIGS. 23A-23B) reveal similar increases in HR, but with a slower onset time observed with delivery from subcutaneously injection, while other biomarkers (e.g., heart rate, SpO₂, and pulse-width distension) exhibit a weak correlation with epinephrine release (FIGS. 24A-24C).

### Long-Term Drug Stability and Opening Through Fully Formed Fibrotic Tissue Layers:

Long-term use may present two challenges. First, dense fibrotic tissue overgrowth layers that form over implanted devices as a consequence of the foreign body response may limit diffusive drug transport and uptake, increasing timelines for drug action in vivo and limiting capabilities in timely intervention for emergency rescue. Second, drug stability considerations demand protection from water ingress, requiring seals capable of withstanding hydrolysis, enzymatic attack, and diffusive water permeation in physiological conditions.

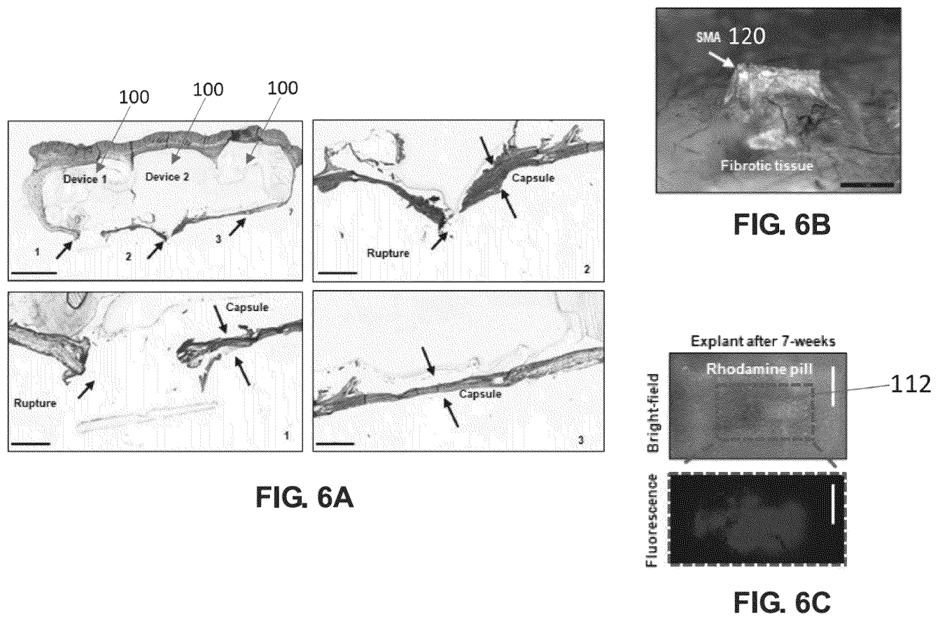

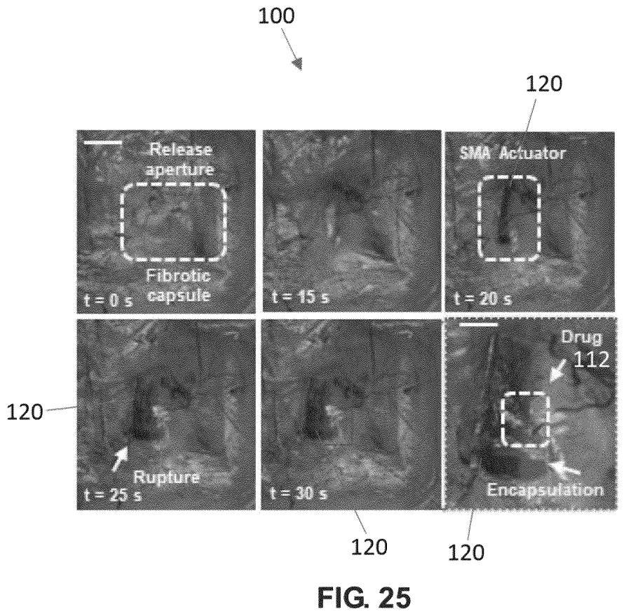

The SMA-based actuation of device 100 may be capable of complete penetration through a fully formed fibrotic capsule in an ex vivo study, as shown in the histology images in FIG. 6A and in the optical image in FIG. 6B. Three distinct sites are demarcated in FIG. 6A: a thinner fibrotic layer (about 300 μm), with complete opening and penetration (site 1, additional images in FIG. 25), a thicker fibrotic layer including subcutaneous fat deposits (about 500 μm), with complete penetration but partial opening (site 2), and an unopened device with an intact fibrotic layer (site 3). The mechanical penetration forces exerted by the SMA are directly proportional to applied thermal actuation powers and accordingly may be tuned to ensure penetration and drug release through thicker fibrotic capsules. The same set of ex vivo experiments also support imaging studies on encapsulated fluorescent pills that confirm a fully intact, stable drug with strong, localized fluorescence after a 7-week implantation period in vivo (FIG. 6C). Taken together, these results represent beneficial capabilities in long-term operation, allowing for the stable storage of emergency drugs, followed by rapid, ejected delivery through fully formed capsules.

### Fabrication and Assembly of Device 100:

(i) Battery/board assembly: Flexible circuits 140 or printed circuit boards (flex-PCBs or fPCBs) were designed on computer-aided design (CAD) modeling software and manufactured at an ISO 13485 registered flexible-circuit board manufacturing facility. Surface-mounted electronic components were attached via reflow soldering. A power source 160, such as a lithium-polymer pouch cells (e.g., PowerStream Inc., Orem, UT) were cast separately in a photocurable adhesive (e.g., Norland Optical Adhesive 63) and attached to bottom layers on assembled circuits 140 (e.g., flex-PCBs). Assembled circuits 140 were then modified with 3-acryloxypropyl trimethoxy silane, (e.g., A-174, Gelest) by submerging in 95% EtOH/5% DI-H₂O. The pH was adjusted to 4.5-5.5 with acetic acid and silane was added to achieve concentrations of 2%. The solutions were stirred at room temperature for 5 minutes before the assembled devices were added and stirred for an additional 3 minutes. Circuits 140 were then cured at 50° C. for about 2 hours, followed by coating with Parylene-C to about a 6 μm final thickness in a chemical vapor deposition chamber. The circuit 140 may be coated with one layer of parylene-C at a thickness of about 6 μm. The circuit 140 may also be coated with a multilayer stack of a deposited alumina/silica (e.g., Alumina, Hafnia, Silica, and/or Polyisobutylene) and parylene C.

(ii) Programming shape memory alloy (SMA) 120 actuators: Custom cut shape-memory alloy (SMA) 120 sheets machined via electrostatic discharged machining to dimensions of about 2.3 mm×15 mm×0.1 mm and with a programmed transition temperature of 35° C., were mechanically confined to their programmed, austenitic curled shape and placed in a >1200° C. flame for about 10 seconds followed by rapid quenching in 21° C. deionized water. The austenitic shape was programmed to bend around a fixed point that served as a hinge 221. The hinge point 221 can be positioned near the middle of the deformable membrane 120 as illustrated in FIG. 2B. The deformable membrane SMA 120 was then flattened and trimmed to the appropriate dimensions via manual metal cutting. The deformable membrane SMA 120 of device 100 may be approximately 3 mm wide by 10 mm long. During trimming, the hinge point 221 was aligned with the base of the pill well (e.g., reservoir 111) to allow for complete opening in a localized manner over the pill well.

(iii) Assembly of integrated wireless devices: Fabrication began with a 3D CAD design, performed on standard 3D modeling software. 3D printing via a stereolithography process on a commercially available 3D printer then yielded initial prototypes for top substrate 170 and bottom substrate 110 that formed the device 100 casing and reservoirs 111 for pill storage. Subsequent optimized designs were constructed from polyamide (e.g., PA-12, Protolabs, Inc., Maple Plain, MN) via a multijet fusion process. The top substrate 170 may include an aperture 171 for release of the pill 112.

Semicircular pills 112 (1 mg, diameter 1 mm) were placed in pill wells (e.g., reservoir 111) structured into bottom substrate 110, with flat pill surfaces attached to SMAs 120 via a thin silicone adhesive (e.g., SilPoxy, Smooth-On Inc., Macungie, Pa) for ejected release following opening. The thin silicone adhesive may assist movement of the pill 112 with the deformable membrane SMA 120 during burst release. A cyanoacrylate adhesive may also provide attachments between the SMAs 120 and substrates 110 and 170 and SMAs 120 and flexible thermal actuators 130, respectively. A thermal insulator (i.e., silicone foam) 150 prepolymer material (e.g., Soma Foama, Smooth-On Inc.,) was separately mixed in a 1:1 w/w ratio and applied to outer surfaces of the flexible thermal actuators 130 via fine needle tips. Substrate 170 may assist with keeping the remaining components of the device 100 (e.g., the deformable membrane 120, the thermal actuator 130, the circuit 140, and the thermal insulator 150) in position after implantation in a human subject.

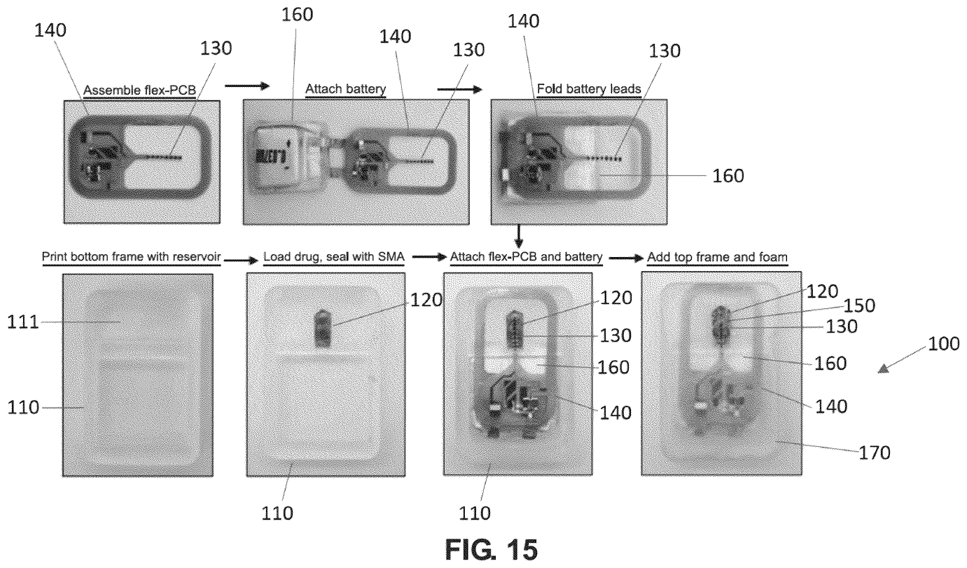

Assembled circuits 140 and batteries 160 were placed in chassis in bottom-substrates 110. Top-substrates 170 were then mechanically mated, with pegs on top surfaces fitted to holes in bottom substrate 110 and attached via a cyanoacrylate-based adhesive. The assembled device surfaces were then modified with 3-acryloxypropyl trimethoxy silane, (e.g., A-174, Gelest) by submerging in 95% EtOH/5% DI-H₂O. The was pH adjusted to 4.5-5.5 with acetic acid and silane was added to achieve concentrations of 2%. Solutions were stirred at room temperature for 5 minutes before the assembled devices were added and stirred for an additional 3 minutes. The devices were then cured, followed by coating with Parylene-C to about a 0.75 μm final thickness in a chemical vapor deposition chamber. Images of the assembly process appear in FIG. 15.

Wired devices 900 used for early studies involved all of the above steps, but flexible actuators 930 connected directly to leads for wired power transfer and devices did not contain batteries or completed flex-PCBs for power harvesting. Examples of the wired device 900 are shown in FIGS. 9A, 9B, and 10. The wired device 900 includes a SMA 920 and a wire 961.

Device sterilization: Devices were dipped in 70% EtOH followed by 30 mins per side of UV sterilization and maintained in sterile conditions prior to implantation.

### Thermal and Mechanical Benchtop Characterization and Modeling:

Thermal and mechanical modeling was performed via 3D steady-state finite element modeling on a commercially available software package (e.g., SolidWorks, Dassault Systemes). Thermal measurements involved attaching miniaturized surface-mounted negative temperature coefficient (NTC) thermistors to circuits 140 (e.g., flexible circuit boards) (thickness <150 μm) and attaching them directly to relevant surfaces, including SMAs 120 and outer, tissue contacting surfaces of assembled emergency rescue devices. Resistance measurements were converted to temperatures via calibration. Mechanical force measurements were performed by suspending flat, programmed SMAs 120 2 mm above planar, resistive pressure sensing pads (e.g., SEN-09375, SparkFun electronics) held in place via silicone spacers. Providing power to flexible thermal actuators 130 on the SMA 120 resulted in shape change and curling, causing a direct force applied on the pressure pads that was read out via a digital multimeter (e.g., NI-4065, National Instruments) and converted to a force measurement via calibration.

### Wireless Energy Harvesting and Triggering Systems:

Two types of wireless triggering mechanisms were evaluated and may be used with the device 100. The first wireless triggering mechanism involved wireless power harvesting system via resonant inductive coupling at a frequency of 13.56 MHz. A wireless NFC-polling system (e.g., X-Nucleo 6, STMicroelectronics) running a customized program on a microcontroller evaluation board (e.g., L476G, STMicroelectronics) served as a primary transmitter coil for benchtop studies and a subset of in vivo studies involving anesthetized, immobilized animals. A specialized cage system (e.g., Neurolux Inc., Skokie Illinois) supported wireless release in awake, freely moving mice, with power transfer mediated via a graphical user interface on a laptop computer. The secondary receiver antennae included L-C circuits, with inductors structured directly into flexible circuit board materials and capacitors screened for optimal power transfer via impedance matching.

The second wireless triggering mechanism assessed the feasibility of triggering via far-field, Bluetooth-Low Energy System-on-Chip (BLE-SoC). BLE-SoC evaluation boards (e.g., a NRF 52832, Nordic Semiconductor) with custom programming supported these studies (FIGS. 16A-16C). A smartphone application with a graphical user interface hosted on a smartphone 1696 with an Android operating system toggled a single general-purpose output (GPIO) pin from its low to high state, causing power supply (e.g., power source 1660) to a flexible thermal actuator array 1630 via the opening of a channel on an n-channel enhancement mode MOSFET 1682.

### Drug Formulation and Pill Production:

Glucagon: The following procedure can be modified for the desired dose of glucagon. Typically, for 4% glucagon formulation, lyophilized hydrochloride salt of glucagon was dissolved in HCl (pH 2.5, 10 mM in H₂O) to prepare a stock solution of 4.2 mM. A portion of this stock solution (272 μL, 1.14 μmol, 4 mg) was diluted in HCl (pH 2.5, 10 mM in H₂O) and added to an aqueous suspension of lactose (96 mg) in an Eppendorf tube. The mixture was thoroughly vortexed and immediately flash frozen in liquid nitrogen. The frozen sample was lyophilized and stored at 4° C. until pressed in pill form.

Insulin: Insulin stock solution was prepared using a similar procedure as above. A portion of the stock solution (164 μL, 689 nmol, 4 mg) was diluted in HCl (pH 2.5, 10 mM in H₂O) and added to an aqueous suspension of lactose (96 mg) in an Eppendorf tube. The mixture was thoroughly vortexed and immediately flash frozen in liquid nitrogen. The frozen sample was lyophilized and stored at 4° C. until pressed in pill form.

Rhodamine: Rhodamine (2.5 mg) was dissolved in PBS 1× (530 μl) to prepare a 10 mM stock solution. This stock solution was serial diluted to 10 μM, 1 μM, 0.1 μM, and 0.01 μM solutions in PBS 1× and used for generating standard curve for UV spectrometric analysis. A portion of 10 μM solution (20 μL) was mixed with 100 mg lactose, flash frozen, and later used for pill production.

Lactose pills: Pills were defined pressing lyophilized powders into 2 mg pills of 2 mm diameter using a pill press and then split into two semicircular 1 mg pills. Pills were defined by pressing lyophilized powders into 2 mg pills of 2 mm diameter and then split into two semicircular 1 mg pills.

### Characterization of Drug Dissolution Rates:

2 mg drug containing pills 112 (e.g., 0.08 mg glucagon and 1.92 mg lactose) were incubated in Eppendorf tubes containing phosphate buffered saline (PBS) solution (200 μl) and were gently shaken at the temperature of 37° C. using a temperature-controlled hot plate shaker. The shaking ensured the dissolved glucagon molecules were uniformly dispersed in the solution. At each testing time point, 20 μl solution was taken from the incubation solution and then 20 μl fresh PBS solution was added to replenish the incubation solution. The testing solution was further diluted twice with a hydrochloride solution (pH of about 2) followed by glucagon concentration measurements via high-performance liquid chromatography (HPLC) (FIG. 21A).

A standard curve for glucagon concentration as a function of absorbance peak area was obtained and shown in FIG. 21B. Several known concentrations of glucagon (x, unit μM) were tested with the HPLC and the corresponding absorbance peak areas (y, A.U.) were measured. In the tests, y=61.88x. This equation yields glucagon concentrations of testing solutions at different time points via measurements of absorbance peak areas. For each time point, the glucagon concentration is provided by equation 1 below:

$$c ( t ) = \frac{y ( t ) \times 2 \times {M}_{glucagon}}{61.88} μ g / L \qquad ( 1 )$$

where M_{glucagon}=3483 g/mol, the molecular weight of glucagon. The glucagon release ratio at each time point (FIG. 21C) is therefore calculated based on equation 2 below:

$$r ( t ) = \frac{c ( t ) \times 2 \times 1 {0}^{- 4}}{80} \qquad ( 2 )$$

HPLC was performed under the following conditions using an Agilent InfinityLab Poroshell 120 (EC-C18, 4.6 mm×100 mm, 2.7 μm particle size) at 25° C., using 0.1% formic acid in water (solvent A) and acetonitrile (solvent B) at 0.6 ml/min. The following linear gradient was employed: (time (min), % B) 0, 0; 10, 0; 25, 80; 30, 100; 35, 0; 45, 0). Glucagon UV absorption was monitored at 280 nm with retention time (Rₜ)=19.2 min.

### A Multi-Reservoir Device:

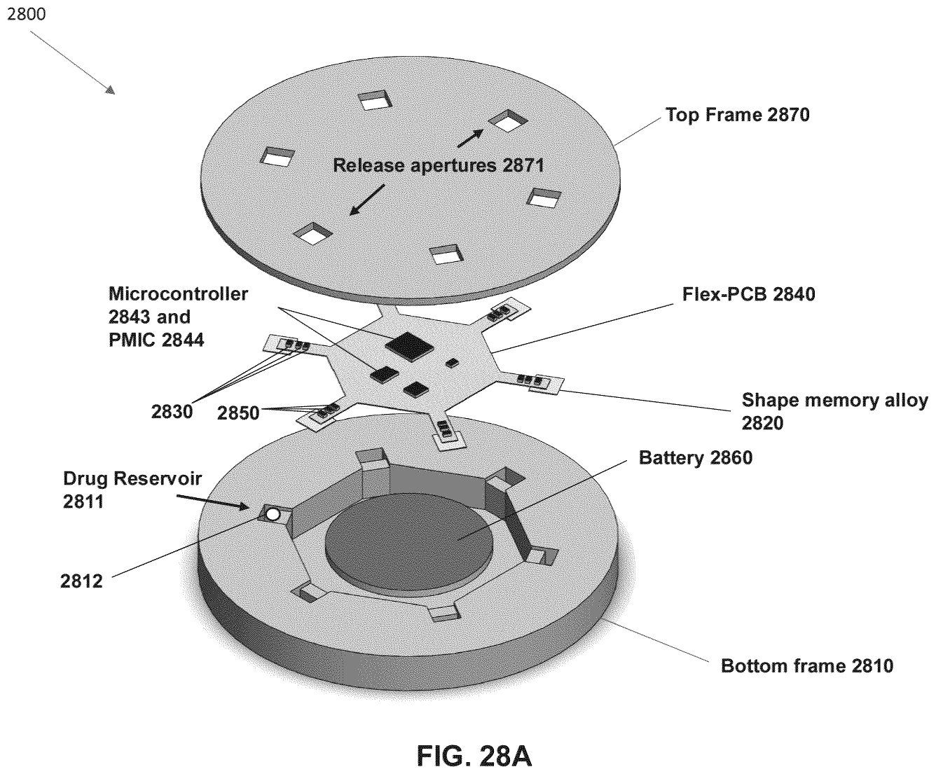

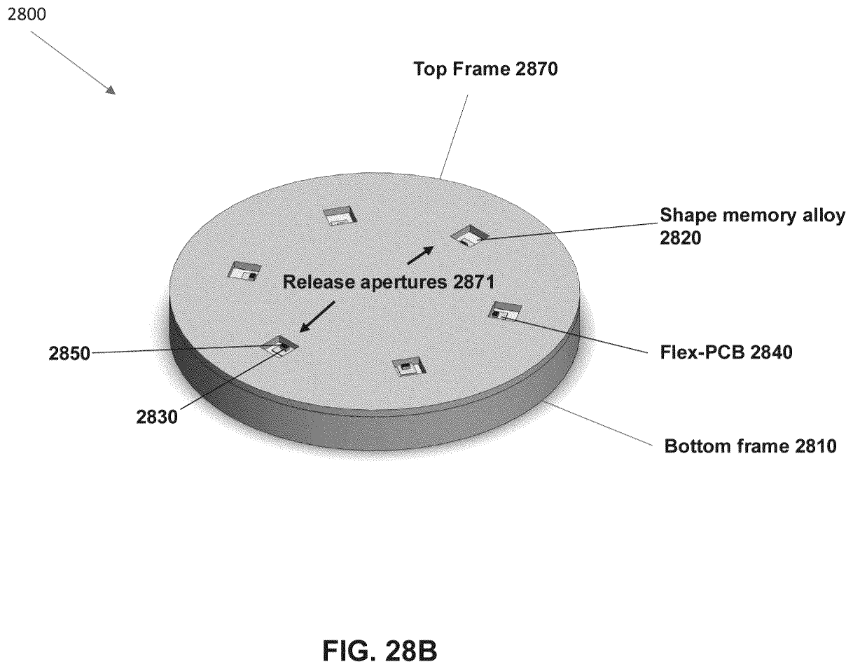

FIGS. 28A and 28B illustrate a multi-reservoir drug delivery system. FIG. 28A shows an exploded view of the multi-reservoir device 2800. FIG. 28B shows an assembled view of the multi-reservoir device 2800. The device 2800 includes a bottom frame or substrate 2810 that includes a plurality of reservoirs 2811 for the storage of a plurality of substances (e.g., a lyophilized drug compressed into pills 2812), with each reservoir 2811 structured inside a recessed feature 2813 for sealing as shown in FIG. 28A. The reservoirs 2811 may be positioned radially around the substrate 2810. The substance 2812 may be substantially in a solid form. The device 2800 may also include a top substrate 2870 that includes apertures 2871 for release of the substance 2812. The top substrate 2870 and the bottom substrate 2810 may be manufactured and attached as described above. Substrate 2870 may assist with keeping the remaining components of the device 2800 (e.g., the deformable membranes 2820, thermal actuators 2830, circuit 2840, and thermal insulators 2850) in position after implantation in a human subject.

The device 2800 also includes a flexible printed circuit board (flex-PCB or fPCB) 2840 as described above. The fPCB 2840 may include one or more surface-mount electronic components including a Bluetooth, Wi-Fi, or cellular antenna and/or a RFID or NFC tag to enable communication with an external device (e.g., a cellular phone or continuous glucose monitor). The device 2800 may also include a microcontroller 2843 and a power management integrated circuit (PMIC) 2844 to enable the multi-reservoir 2811 drug release. The fPCB 2840 may cover a portion of the reservoirs 2811 as shown in FIG. 28B. The device 2800 may also include a power source 2860 (e.g., a battery) as described above.

The microcontroller 2843 (e.g., aATTINY-25V, Atmel) supports active release from multiple reservoirs 2811. A custom program loaded onto the microcontroller 2843 via an Arduino-based interface via an MLF-20 board allows for sequential release from multiple reservoirs 2811. A regulated output (3.3V) provides power to the microcontroller 2843. The microcontroller 2843 can be programmed with a state-counter, with each state corresponding to a separate GPIO pin. The microcontroller 2843 can be programmed to immediately enter a low-power, deep-sleep mode following programming. A voltage supply (e.g., V>2 V) to a specialized interrupt (INT) pin ends the deep-sleep mode and enters the microcontroller 2843 into a subroutine comprising toggling a GPIO pin corresponding to the existing state on the state counter to its high state for 60 s, followed by reverting it back to its low state, incrementing the state-counter and then re-entering deep sleep mode. In this way, a subsequent wake-up event toggles power to the next GPIO pin. Toggling the GPIO pin to high opens a channel on a MOSFET that allows current to flow from the regulated voltage supply to a flexible thermal actuator 2830, resulting in deformable membrane SMA 2820 shape change and drug release.

The device 2800 may also include a plurality of thin, flexible linear heating arrays (e.g., a plurality of thermal actuators 2830) as described above. Each reservoir 2811 may have a corresponding thermal actuator 2830 as shown in FIG. 28A. As described above, each thermal actuator 2830 may include an array of linearly arrayed surface-mounted resistive elements electrically connected in series on the flex-PCB 2840. The resistive elements may also be attached via reflow soldering onto pads as described above. Each of the resistive elements of each of the thermal actuators 2830 may also be thermally insulated with a thermal insulator 2850 (e.g., a silicone foam) as described above.

The device 2800 is compatible with peptides and proteins (e.g., glucagon and insulin), small molecules (e.g., epinephrine) and fluorescent dyes (e.g., Rhodamine-C) and may demonstrate a therapeutic benefit. Each of these classes of drugs may be formulated in the manner described above. Additionally, each pill 2812 may also be prepared as described above. Each pill 2812 may be sealed in an individual reservoir 2811 via a deformable membrane 2820 (e.g., a nickel-titanium SMA membrane). The SMA 2820 may operate as described above, such that when the SMA 2820 temperature is raised above the SMA's transition temperature, the SMA 2820 deforms into an open configuration thereby opening one reservoir 2811 of the device 2800 and ejecting one pill 2812. The device 2800 may hold a plurality of therapeutically effective doses of a drug in the form of a plurality of pills 2812, wherein each reservoir 2811 contains at least one pill 2812. Each pill 2812 may be a circular pill, a rectangular pill, and/or any other suitable shape for the device 2800.

The device 2800 may also include an edge-sealant including a polymer (e.g., Parylene-C, Polyisobutylene, Polyimide, Polytetrafluoroethylene, Styrene-Ethylene-Butylene-Styrene thermoplastic elastomer (SEBS), Polystyrene, Polyethylene, and/or another low-permeability polymer) and a hydrophobic wax (e.g., beeswax, Paraffin, Candelilla) as described above. The sealant may seal each substance 2812 in an individual reservoir 2811 to provide complete sealing in the closed configuration shown in FIG. 28B.

In operation, the device 2800 may allow for the multiple triggered, individual release of a therapeutically effective dose of a drug. The device 2800 may be implanted or injected subcutaneously into a patient. The device 2800 may then be triggered via an external device (e.g., a cellular phone or continuous glucose monitor) to open at least one of the plurality of reservoirs 2811, ejecting at least one of the substances 2812 (e.g., a pill). Opening of the device 2800 may involve heating at least one of the deformable membranes 2820 above its transition temperature using at least one of the thermal actuators 2830.

Closed-Loop Integration with Continuous Glucose Monitor:

An overview of the system is shown in FIG. 20. A Dexcom G6 continuous glucose monitor (CGM) 496 was used. Data was transferred from the CGM 496 to a cloud service can be accessed via a Python-based Application Programming Interface (API), pydexcom (developed by GitHub user gagebenne). A custom program designed for hypoglycemia detection and drug release, glucagon.py, used pydexcom to receive real-time CGM data, and detect acute hypoglycemic events. Successive glucose measurements <54 mg/dl resulted in the toggling of a GPIO pin from a low to a high state on a microcontroller, which then activated a primary coil that resulted in a harvested voltage at the secondary coil and subsequent glucagon release. The custom program for this application, glucagon.py was hosted on a microprocessor and is available at: https://github.mit.edu/athenaw/automatic_glucagon.

Cron commands, available on Linux-based operating systems to run background scripts, allowed the microprocessor to continuously run a script to check for glucose values. The cron command is written in cron_command.txt and is given by:

* * * * * cd cygdrive c Users wanga uropfall 2022 automatic_glucagon && cygdrive c Users wanga/AppData Local/Microsoft WindowsApps python.exe glucagon.py

As written, every minute, the system was instructed to go to the folder “/cygdrive/c/Users/wanga/urop/fall_2022/automatic_glucagon”, where it executed the custom program glucagon.py. The sampling rate (1/minute) is greater than the sampling rate of the CGM (⅕ minutes) and ensures that no values are missed. Glucagon.py was designed to connect to Dexcom accounts and microcontrollers to get up-to-date blood glucose measurements and write them to text (e.g., JSON) files. Real-time computations then resulted in setting a selected microcontroller pin to low if BG >55 mg dl⁻¹ and high if BG<55 mg dl⁻¹. Setting the selected pin to high then activated a primary transmitter coil that resulted in power transfer to the emergency rescue device and subsequent opening.

Depending on the value of the blood glucose measurement, the system will write low or high to pin 13 of the Arduino. The threshold for a low blood glucose measurement was set to 55 mg/dl. This means that if the glucagon.py script receives a value that is below 55 mg/dl, it will write a 1 to pin 13 of the Arduino. Testing involved submerging the CGM 496 probe and emergency rescue device in a glucose-DI-H₂O solution with a starting concentration of 80 mg ml⁻¹, in a container placed in proximity to a primary transmitter coil. Continuous infusion of DI-H₂O at 1 ml/min into the glucose-DI-H₂O solution via a calibrated syringe pump and gentle stirring resulting in dilution of the glucose solution, until the glucose concentration went below 55 mg dl-1, the programmed threshold for hypoglycemia, resulting in automatic release of Rho-Lac from the emergency rescue device via the process described above. Aspiration of 100 μl liquid samples at 5-minute intervals and readout via a spectrophotometer resulted in concentration measurements for Rhodamine in the solution.

### Accelerated Rate Testing and Screening of Encapsulation Materials:

FIG. 7A shows an image of a simplified device 700 with a pill well 711 and recessed area for the SMA 720. The simplified device 700 created using SolidWorks 2021 before being printed in a photocurable resin 713 on a Form 3 printer. To visualize changes in pill quality over time, an optically transparent ‘window’ through the back of the device was created using a layer of UV curable optical adhesive or transparent resin 713 (e.g., Norland Optical Adhesive, 63) opposite the pill well 711 and SMA 720 recess. The transparent resin 713 may also include a drop of UV-cured epoxy on the back to create a smoother surface and increase visibility through the transparent resin 713. A 1 mg Rho-Lac pill was encapsulated in each sample according to the wax encapsulation method described above. Unprogrammed SMAs supported accelerated rate testing at elevated temperature of 50° C. Devices were then coated in parylene at thicknesses of about 0.75 um, about 1.50 um, or about 3.00 um. Each device was then placed SMA-down on a 1% agarose gel 715 and stored at 50° C. Here, accelerated rate testing can be modeled as an Arrhenius reaction rate function, and is given by equation 3:

$$k = A {e}^{- {E}_{a} / R T} \qquad ( 3 )$$

Where A is an empirically determined constant, Ea is the activation energy, T is the temperature of the reaction (in Kelvin) and R is the universal gas constant. The ratio between reactions proceeding at physiological temperatures (T₁=37° C.=310 K) and elevated temperatures (T2=50° C.=323 K) is given by equation 4:

$$\frac{{k}_{50 ° C .}}{{k}_{37 ° C .}} = exp [ \frac{{E}_{a}}{R} ( \frac{1}{323} - \frac{1}{310} ) ] ∼ {2}^{1.3} \qquad ( 4 )$$

Accordingly, 30 days at 50° C. corresponds to ˜50×2^{1.3}=123 days at 37° C. Fluorescence imaging (4× magnification, RFP channel, excitation at 532 nm, emission at 588 nm, exposure time 500 milliseconds) revealed drug transport induced by water ingress. Image analysis software facilitated two types of computations: (i) normalized mean fluorescence, and (ii) total area of fluorescence. Normalized mean exposure was assessed by sampling fluorescence at three locations in each image, averaged, and baseline corrected for the mean fluorescence of a dry pill. This was then converted to a percentage of maximum possible mean fluorescence. Total fluorescent area was determined by converting images to binary with an appropriate and consistent threshold. The area of fluorescence was then calculated as a percentage of total image area.

### In Vitro Release Characterization

In vitro release profiles were tested in two systems: 1) saline solutions and 2) hydrogel tissue mimics. Release in saline solutions allowed for quantification of concentration changes owing to drug release and involved completely submerging assembled devices in 3-5 ml phosphate-buffered saline (pH 7.4), with gentle magnetic stirring. 100 μl saline was extracted for each measurement period, at intervals of 10 minutes prior to release and at intervals of 2 minutes following release and pipetted into specialized transparent containers. Optical measurements in a spectrophotometer based on calibrations obtained from standard curves then quantified concentrations of Rhodamine-C, Insulin and Glucagon, respectively, depending on the study performed.

Release in hydrogels allowed for the study of drug transport following ejected release in tissue-mimic systems. Devices were cast and completely submerged in 1% Agarose hydrogel in transparent well plates, followed by either wired or wireless release. Time-series optical bright field and fluorescence imaging then captured diffusion profiles. A subset of measurements involved directly placing Rhodamine-C-Lactose drug pills in 1% Agarose, followed by imaging, to determine diffusion profiles.

### Surgical Device Implantation Procedures:

(i) Preparation: Mice were anesthetized under continuous flow of 1-4% isofluorane with oxygen at 0.5 L/min. A shaver was used to remove hair in the regions that received implants. The entire shaved area was aseptically prepared with scrubbing with povidine followed by rinsing with 70% alcohol over multiple cycles. A final skin paint with povidine was applied. All devices were sterilized prior to cell loading and via ethanol and UV and maintained in sterile conditions until implant.

(ii) Surgical procedure: A sharp surgical scissor was used to cut a 0.5-0.75 cm incision through the skin. Subsequently, a subcutaneous pocket of about 1 cm×1 cm was created via blunt dissection and devices were carefully placed inside the pocket using a pair of blunt tweezers, with care taken not to touch the membrane. The membrane side was inserted facing down onto the muscle bed, while the cathode side of the device faced the skin. The skin layer was closed via sutures. Blood and tissue debris were removed from the surgical instruments between procedures and the instruments. After the surgery, animals were placed back in cages on a heat pad or under a heat lamp and monitored until they came out of anesthesia.

(iii) Intraoperative care: Animals were kept warm via heating pads; eyes were hydrated with sterile ophthalmic ointment during the period of surgery. Care was taken to avoid wetting the surgical site excessively to avoid hypothermia. The respiratory rate was monitored continuously throughout the surgical period.

### In Vivo Imaging:

Animals were anesthetized via isoflurane and placed in an in vivo imaging system (IVIS). Baseline fluorescent measurements tuned to emission peaks of cy7 (exposure time 3 seconds, 640 nm<wavelength<840 nm) established pre-release fluorescence profiles. A primary antenna placed in close proximity to the device for about 1 minute induced wireless drug release. Sequential imaging following release at intervals of about 5 minutes then allowed for imaging of fluorescence signals resulting from drug dissolution and diffusion. 35 minutes following release, mice were euthanized and the organs were then explanted and imaged using the same parameters as above to characterize the biodistribution of cy-7 labeled insulin and glucagon.

### Blood Glucose (BG) Measurements:

0.5 μl volumes of blood were collected via tail-pricks with 25-27G and measured with a commercial handheld blood glucose meter (e.g., a Clarity, BG1000). The upper limit of measurements is 600 mg/dl on these systems and values above this were recorded as 600 mg/dl for analysis. Non-fasting values were recorded for 7-10 days following implantation, after which BG values were recorded following a 2.5 hour fast. Mice were weighed before and after fasting periods. Measurements in experimental groups (O₂, Non O₂ controls) were recorded in triplicate to account for any variability in readings and averaged.

### In Vivo Glucagon Release Studies:

(i) Post-fasting studies: These studies were performed 2 days after device implantation in STZ-induced diabetic BL6 mice. Mice were fasted overnight followed by a BG measurement to establish post-fast baseline values. Device release was triggered by placing the mice in a wireless primary transmitter coil wrapped around a standard mouse enclosure for 3-5 minutes, operated at 10 W and 100% duty cycle. BG values recorded immediately following release served as t=0 minutes measurements. Subsequent BG measurements were taken at 5, 10, 15, 20, 30-, 40-, 50-, and 60-min time points, respectively. All release and BG measurements were performed on awake, freely moving mice. Following the 60-minute time point, mice were euthanized, and devices were explanted and imaged to confirm complete opening and drug dissolution.

(ii) Post-insulin bolus: Mice were prepared by taking a pre-fasting BG measurement, fasted for 3 hours, and measured again. A 200 μL bolus of 2% insulin in sterile saline was injected subcutaneously and BG measurements were taken immediately after at 0, 30, 60, and 90 minutes or until BG measurements fell below 100 mg/dL. Drug release was triggered in a wireless primary transmitter coil wrapped around a standard mouse enclosure for 3-5 minutes, operated at 10 W and 100% duty cycle. Immediately following release a BG measurement was taken and set as the 0 min time point. BG values recorded immediately following release served as t=0 minutes measurements. Subsequent BG measurements were taken at 5, 10, 15, 20, 30-, 40-, 50-, and 60-min time points, respectively. As above, all release and BG measurements were performed on awake, freely moving mice. Following the 60-minute time point, mice were euthanized, and devices were explanted and imaged to confirm complete opening and drug dissolution.

### In Vivo Epinephrine Release Studies:

In vivo epinephrine release involved studies on anesthetized adult, male, healthy C57/BL6J mice. Mice were implanted with emergency rescue devices containing epinephrine doses of 1.4 mg/kg via the surgical procedures described above and allowed to recover for a 3-day period. On day 3, mice were carefully anesthetized via the flow of 1% isoflurane, and hair around the dorsal neck region was removed via the application of a depilatory cream (e.g., Nair). A collar-mounted optical probe (e.g., a MouseOx, Starr Life Science, Oakmont, PA) was then used to track heart rate. Additional biomarkers measured by the probe included oxygen saturation (SpO₂), pulse-width distension (a measure of blood flow) and breath rate. Animals were monitored until their heart rate reached steady values of 380-400 bpm. A primary transmitter antenna was then brought into close proximity for 2-5 minutes to induce wireless release. Anesthetized animals were then monitored via optical probe measurements for 30 minutes, followed by euthanasia and explantation to evaluate opening and drug dissolution.

### Histology and Imaging of Opening Through Fibrotic Capsules:

Simplified drug reservoir devices containing Rho-Lac pills with SMA-sealants and flexible-thermal actuators with pads for direct attachment via wired leads were implanted into subcutaneous sites in adult, male C57BL6J mice. Following a 7-week implantation period, devices were explanted along with fully formed fibrotic capsules. Small sections of fibrotic tissue around regions with bond pads for wired lead connections were excised with sharp blades to expose pads. Clip-on wires then established connections to wired, DC power sources. The application of 5V resulted in complete opening, as captured by optical images shown in FIGS. 6A-6C. Samples were then immersed in a fixative (e.g., 4% paraformaldehyde) for 20 minutes at room temperature, followed by multiple washes with PBS. Devices were then stored in 70% ethanol, followed by embedding in paraffin wax, sectioning, staining, and imaging via standard histological methods.

### An Injectable Drug Delivery System

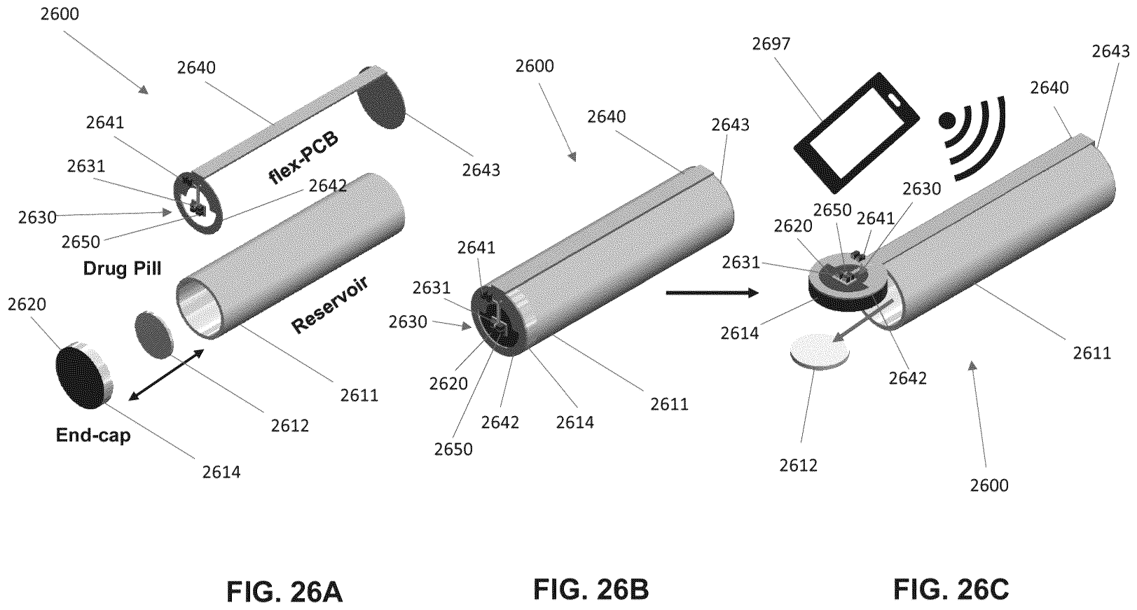

FIGS. 26A-26C illustrate an injectable drug delivery system that includes a thermally actuated SMA membrane for burst release of a solid, stable drug. FIG. 26A shows an exploded view of an injectable device 2600. The injectable device 2600 may be approximately 3 cm to 6 cm in length and approximately 5 mm in diameter. The injectable device 2600 includes a cylindrical shaped reservoir 2611 for the storage of a lyophilized drug compressed into pills 2612. The reservoir 2611 is sealed with an end cap 2614. The end cap 2614 may be made of a deformable membrane 2620 to enable opening of the injectable device 2600. Alternatively, the end cap 2614 may be made of a thin, polymeric sealant (e.g., parylene-C at a thickness of about 1 μm or less).

The injectable device 2600 also includes a circuit 2640. The circuit 2640 may include a flexible printed circuit board (fPCB) as described above. The circuit 2640 may include one or more surface-mount electronic components 2641 including a Bluetooth, Wi-Fi, or cellular antenna and/or a RFID or NFC tag to enable communication with an external device (e.g., a cellular phone 2697 or continuous glucose monitor). The circuit 2640 may be positioned on the outside of the reservoir 2611 as shown in FIGS. 26A-26C. The circuit 2640 may cover a portion of the reservoir 2611. The circuit 2640 may include a front portion 2643 and an end portion 2642 such that the reservoir 2611 is sealed on both ends by the circuit 2640 as illustrated in FIG. 26B. The end portion 2642 may be attached to the outside of the end cap 2614 to create a fluid-tight seal.

The device 2600 may also include a thin, flexible linear heating array (e.g., thermal actuator 2630) as described above. The thermal actuator 2630 may be positioned on the front portion 2643 of the circuit 2640 as shown in FIG. 26A. As described above, the thermal actuator 2630 may include an array of linearly arrayed surface-mounted resistive elements 2631 electrically connected in series on the circuit 2640. The resistive elements 2631 may also be attached via reflow soldering onto pads as described above. The resistive elements 2631 of the thermal actuator 2630 may also be thermally insulated with a thermal insulator 2650 (e.g., a silicone foam) as described above.

The device 2600 is compatible with peptides and proteins (e.g., glucagon and insulin), small molecules (e.g., epinephrine) and fluorescent dyes (e.g., Rhodamine-C) and may demonstrate a therapeutic benefit. Each of these classes of drugs may be formulated in the manner described above. Additionally, the pill 2612 may also be prepared as described above. The pill 2612 may be sealed in the reservoir 2611 via a deformable membrane 2620 (e.g., a nickel-titanium shape memory alloy (SMA) 2620. The SMA 2620 may operate as described above, such that when the SMA 2620 temperature is raised above the transition temperature, the SMA 2620 is programed into an open configuration thereby opening the device 2600 and ejecting the pill 2612, as illustrated in FIG. 26C. The device 2600 may hold one therapeutically effective dose of a drug in the form of pill 2612. The pill 2612 may be a circular pill, a rectangular pill, and/or any other suitable shape for the device 2600.

The device 2600 may also include an edge-sealant including a polymer (e.g., Parylene-C, Polyisobutylene, Polyimide, Polytetrafluoroethylene, Styrene-Ethylene-Butylene-Styrene thermoplastic elastomer (SEBS), Polystyrene, Polyethylene, and/or another low-permeability polymer) and a hydrophobic wax (e.g., beeswax, Paraffin, Candelilla) as described above. The sealant may seal the front portion 2643 and the end portion 2642 of the circuit 2640 to the reservoir 2611 to provide complete sealing in the closed configuration shown in FIG. 26B. The sealant may also seal the end cap 2614 to the reservoir and the end portion of the circuit 2640 to provide complete sealing in the closed configuration shown in FIG. 26B.

In operation, the sealed device 2600 may be injected through the skin. Preferably the device 2600 is injected subcutaneously into a patient's arm. The device 2600 may then be triggered to open after injection, ejecting the pill 2612 via an external device (e.g., a cellular phone 2697 or continuous glucose monitor) as shown in FIG. 26C. Opening of the device 2600 may involve heating the SMA 2620 above its transition temperature using the thermal actuator 2630.

### A Wearable Drug Delivery System

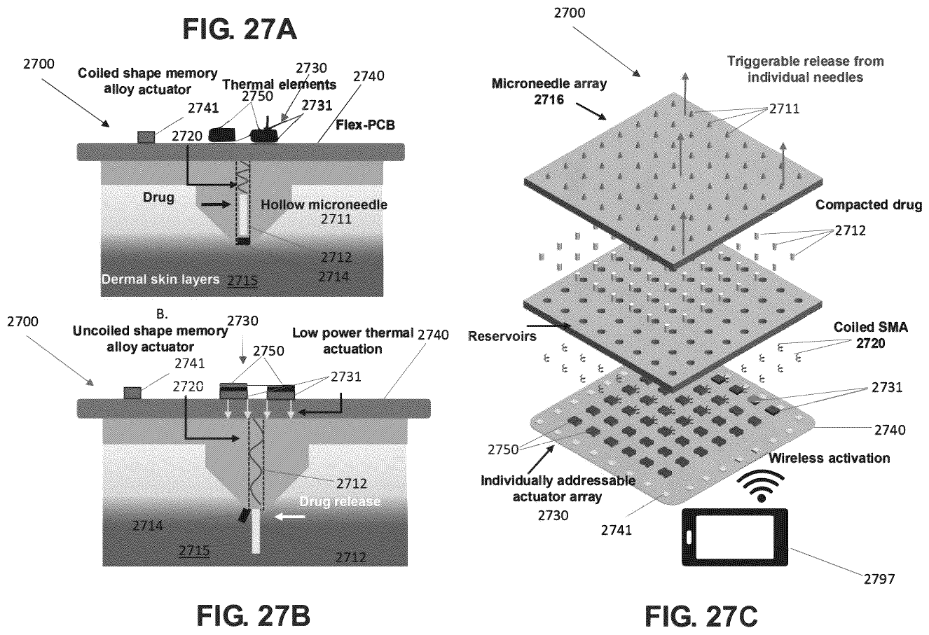

FIGS. 27A and 27B illustrate a wearable drug delivery system that includes a thermally actuated SMA coils for burst release of solid, stable drug from microneedle cavities. FIG. 27A shows an image of a wearable device 2700. The device 2700 includes a hollow microneedle 2711 for the storage of a compacted drug 2712, like the reservoir of device 100. The hollow microneedle 2711 is sealed with an end cap 2714. The end cap 2714 may be made of a thin, polymeric sealant (e.g., parylene-C at a thickness of about 1 μm or less), which may rupture upon the deformation of the deformable membrane 2720. Alternatively, the end cap 2714 may be made of a deformable membrane 2720 to enable opening of the device 2700 as described above. The device 2700 also includes a circuit 2740. The circuit 2740 may include a flexible printed circuit board (fPCB) as described above. The circuit 2740 may include one or more surface-mount electronic components 2741 including a Bluetooth, Wi-Fi, or cellular antenna and/or a RFID or NFC tag to enable communication with an external device (e.g., a cellular phone 2797 or continuous glucose monitor). The hollow microneedle 2711 may extend from the circuit 2740 towards a human subject's dermal skin layer 2715 such that when the hollow microneedle 2711 opens the drug 2712 is injected subcutaneously into the dermal skin layer 2715, as shown in FIG. 27A.

The device 2700 may also include a thin, flexible linear heating arrays (e.g., thermal actuator 2730) as described above. The thermal actuator 2730 may be positioned on the distal side of the circuit 2740 (i.e., away from the dermal skin layer 2715) as shown in FIG. 27A. As described above, the thermal actuator 2730 may include an array of linearly arrayed surface-mounted resistive elements 2731 electrically connected in series on the circuit 2740. The resistive elements 2731 may also be attached via reflow soldering onto pads as described above. The resistive elements 2731 of the thermal actuator 2730 may also be thermally insulated with a thermal insulator 2750 (e.g., a silicone foam) as described above.

The compacted drug 2712 may be injected into the dermal skin layer using a deformable membrane 2720 (e.g., shape memory alloy (SMA)). The deformable membrane 2720 is shown in its closed position (i.e., a coiled position) in FIG. 27A. The deformable membrane 2720 extends into the hollow microneedle 2711. The deformable membrane 2720 may operate as described above, such that when the deformable membrane 2720 temperature is raised above the transition temperature, the deformable membrane 2720 is programed into an open configuration (i.e., uncoiled position) thereby pushing the hollow microneedle 2711 into the dermal skin layer 2715, opening the device 2700, and ejecting the compacted drug 2712 into the dermal skin layer 2715 as illustrated in FIG. 27B.

The device 2700 is compatible with peptides and proteins (e.g., glucagon and insulin), small molecules (e.g., epinephrine) and fluorescent dyes (e.g., Rhodamine-C) and may demonstrate a therapeutic benefit. Each of these classes of drugs may be formulated in the manner described above. The drug may be a lyophilized drug that is compressed into a pill as described above. The device 2700 may hold one therapeutically effective dose of a drug in the form of pill 2712. The pill 2712 may be a circular pill, a rectangular pill, and/or any other suitable shape for the device 2700.

The device 2700 may also include an edge-sealant including a polymer (e.g., Parylene-C, Polyisobutylene, Polyimide, Polytetrafluoroethylene, Styrene-Ethylene-Butylene-Styrene thermoplastic elastomer (SEBS), Polystyrene, Polyethylene and/or another low-permeability polymer) and a hydrophobic wax (e.g., beeswax, Paraffin, Candelilla) as described above. The sealant may seal the end cap 2714 to the hollow microneedle reservoir 2711 to provide complete sealing in the closed configuration shown in FIG. 27A.

The device 2700 may also include multiple hollow microneedles 2711 that form a microneedle array 2716 as shown in FIG. 27C. The microneedle array 2716 may allow for triggerable release from each hollow microneedle 2711 of the microneedle array 2716. Each hollow microneedle 2711 of the microneedle array 2716 may include a compacted drug 2712, a coiled deformable membrane SMA 2720, a thermal actuator 2730, and resistive elements 2731 as described above. Each thermal actuator 2730 of the microneedle array 2716 may also include a thermal insulator 2750 (e.g., a silicone foam) on top of the resistive elements 2731 as described above. The device 2700 may be able to hold a large number of therapeutically effective doses of the drug 2712. Each compacted drug 2712 may be the same drug (i.e., to allow for the administration of multiple doses of the same drug over time). Alternatively, each compacted drug 2712 may be a different drug such that the microneedle array 2716 allows for triggerable release of multiple drugs. The device 2700 also includes an individually addressable thermal actuator array 2730. The thermal actuator array 2730 includes an array of linearly arrayed surface-mounted resistive elements 2731 electrically connected in series on a circuit 2740. The resistive elements 2731 may also be attached via reflow soldering onto pads as described above.

In operation, the device 2700 may be a wearable patch (e.g., like a band-aid) that may inject a compacted drug 2712 (or drugs) subcutaneously through a human subject's dermal skin layer 2715. The device 2700 may be located anywhere on a human subject's dermal skin layer 2715, for example, on an arm or a leg. The device 2700 may be triggered to open, ejecting the compacted drug 2712 via an external device (e.g., a cellular phone 2697 or continuous glucose monitor) as shown in FIG. 26C. Heating the deformable membrane SMA 2720 above its transition temperature using the thermal actuator 2730 may lead to the uncoiling of the deformable membrane SMA(s) 2720 which pushes the compacted drug 2712 into the dermal skin layer 2715 via the hollow microneedle(s) 2711. The external device (e.g., cellular phone 2697 or continuous glucose monitor) may activate one or more hollow microneedle 2711 if the device 2700 includes a microneedle array 2716.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A device, comprising:
a substrate including a reservoir to hold a substance;
a deformable membrane disposed on the substrate and forming a fluid-tight seal over the reservoir;
a thermal actuator mechanically and thermally coupled to the deformable membrane;
a thermal insulator, thermally coupled to the thermal actuator, to channel heat from the thermal actuator to the deformable membrane and to reduce dissipation of heat from the thermal actuator into tissue surrounding the device;
a circuit operably coupled to the thermal actuator, the circuit configured to receive a control signal and, responsive to the control signal, heat the thermal actuator such that the thermal actuator deforms the deformable membrane to open the fluid-tight seal and effect release of the substance from the reservoir; and
a power source configured to provide power to the thermal actuator and the circuit.

2. The device of claim 1, wherein the device is sized to be injectable or implantable in a human subject.

3. The device of claim 1, wherein a first end of the deformable membrane is fixedly attached to the substrate and a second end of the deformable membrane is removably attached to the substrate, such that the deformable membrane deforms by bending such that the second end of the deformable membrane moves away from the substrate.

4. The device of claim 3, wherein the substance is substantially in solid form.

5. The device of claim 4, wherein the substance is coupled to the deformable membrane, such that deformation of the deformable membrane effects decoupling of the substance from the deformable membrane and subsequent release of the substance from the reservoir.

6. The device of claim 1, wherein the substance includes at least 30 μg of an active pharmaceutical ingredient.

7. The device of claim 6, wherein the active pharmaceutical ingredient comprises at least one of glucagon, insulin, or epinephrine.

8. The device of claim 1, wherein circuit includes a flexible printed circuit board (fPCB) fixedly coupled to the thermal actuator, the fPCB having a flexural rigidity selected to withstand a bending strain experienced by the fPCB and/or the thermal actuator during the deformation of the deformable membrane to remain coupled to the thermal actuator.

9. The device of claim 1, wherein the substrate is a first substrate and further comprising:
a second substrate coupled to the first substrate to form an enclosure containing the thermal actuator, the thermal insulator, the circuit, and the power source.

10. The device of claim 1, wherein the thermal insulator comprises a silicone foam.

11. The device of claim 1, further comprising a sealant disposed on the deformable membrane to form at least a portion of the fluid-tight seal.

12. The device of claim 11, wherein the sealant comprises a multi-layer film comprising a polymer and a hydrophobic wax.

13. The device of claim 1, wherein the control signal is provided by a cellular phone or a continuous glucose monitor.

14. The device of claim 1, wherein:
the reservoir is one of a plurality of reservoirs to hold a plurality of substances;
the deformable membrane is one of a plurality of deformable membranes; and
the thermal actuator is one of a plurality of thermal actuators mechanically coupled to the plurality of deformable membranes.

15. The device of claim 14, wherein the plurality of substances comprises individual doses of an active pharmaceutical ingredient.

16. A method of administering a substance to a human subject with a device comprising a substrate forming a reservoir holding the substance, a deformable membrane disposed on the substrate and forming a fluid-tight seal over the reservoir, a thermal actuator mechanically and thermally coupled to the deformable membrane, a thermal insulator thermally coupled to the thermal actuator, and a circuit operably coupled to the thermal actuator, the method comprising:
disposing the device subcutaneously in the human subject;
actuating the circuit to heat the thermal actuator;
deforming the deformable membrane with heat from the thermal actuator so as to decouple the substance from the reservoir; and
delivering the substance into the human subject via the decoupling of the substance from the deformable membrane.

17. The method of claim 16, wherein disposing the device subcutaneously comprises injecting the device.

18. The method of claim 16, wherein disposing the device subcutaneously comprises implanting the device.

19. The method of claim 16, wherein the deforming the deformable membrane produces a force sufficient to tear through fibrotic tissue surrounding the device.

20. The method of claim 16, wherein the actuating of the circuit is in response to a signal from at least one of a cellular phone or a continuous glucose monitor.
